(19) United States
(12) Patent Application Publication
(10) Pub. No.: US 2015/0004147 A1
(43) Pub. Date: Jan. 1, 2015

(54) Lactose Compositions With Decreased Lactose Content

(71) Applicant: Ritter Pharmaceuticals, Inc., Los Angeles, CA (US)

(72) Inventor: Andrew J. Ritter, Los Angeles, CA (US)

(21) Appl. No.: 14/171,262

(22) Filed: Feb. 3, 2014

Related U.S. Application Data
(63) Continuation of application No. 12/996,975, filed on Feb. 28, 2011, now abandoned.
(60) Provisional application No. 61/075,669, filed on Jun. 25, 2008.

(51) Int. Cl. A61K 31/7016; A61K 35/74; A61K 31/732; A61K 31/717; A61K 31/736; A23G 3/42; A61K 31/702

(52) U.S. Cl. CPC A61K 31/7016; A23G 3/42; A61K 35/747; A61K 35/745; A61K 31/702; A61K 31/717; A61K 31/736; A61K 31/732; A61K 2035/115
USPC 424/93.45; 514/53; 424/93.4

(57) ABSTRACT

The invention provides methods and compositions for treating symptoms associated with lactose intolerance and for overall improvement in gastrointestinal health. Described herein are methods and compositions for improving overall gastrointestinal health or for decreasing symptoms of lactose intolerance by administering to an individual a lactose composition with decreased lactose content in combination with effective amounts of prebiotics and/or probiotics.

### CROSS-REFERENCE

This application is a continuation application of Ser. No. 12/996,975, filed Jun. 25, 2009, which is a National Stage Entry of PCT/US09/03834, filed Jun. 25, 2009, which claims the benefit of U.S. Provisional Application No. 61/075,699, filed Jun. 25, 2008, each of which is incorporated herein by reference in its entirety.

### BACKGROUND OF THE INVENTION

According to several sources, there are 30 to 50 million people in the world who are lactose intolerant. In the 1960's and 1970's, it was reported that 70% of the adults in the world had lactose intolerance. In 1995, it was reported that 75% of the adults in the world and 25% of the adults in the U.S. were categorized as being lactose intolerant. In 1994, it was reported that 75% of African Americans and Native Americans and 90% of Asian Americans had lactose intolerance. It has also been reported that 30% of adults who are mostly North Western and North American descendants of the Europeans, have adapted to high lactase activity into adulthood. Research concludes that this adaptation is genetically controlled, permanent, and related to a long tradition of milk and milk product consumption in these regions of the world.

Lactose intolerance is the inability to digest significant amounts of lactose, a major natural sugar found in milk and milk products of all mammals. Lactose intolerance is caused by a shortage of the enzyme lactase, which is produced by the cells that line the small intestine and is essential to lactose digestion. Lactase breaks down the lactose, a disaccharide, into two simpler forms of sugar called glucose and galactose, which are then transported across the cell membrane and absorbed into the bloodstream. If lactase is not present, or not present in sufficient levels, excess undigested lactose passes through the small intestines into the large intestine where it is fermented by a bacteria in the colon (“colonic flora”, “gut flora”, or “intestinal flora”). The fermentation of the lactose in the large intestine produces hydrogen and methane which can lead to bloating, gas, and diarrhea. These symptoms are caused by a very low activity of lactase in the intestines and are found in individuals who are lactose intolerant. Not all people deficient in lactase have the symptoms commonly associated with lactose intolerance, but those who do are said to have lactose intolerance.

If an individual suspects that he or she has lactose intolerance, it is potentially harmful for him or her to restrict his or her diet since it can result in a nutrition shortage or a failure to detect a more serious disease. Milk and other dairy products are major sources for nutrition in the basic American diet. The primary nutrients in milk are protein, calcium, riboflavin, vitamin A, and vitamin D. Calcium is an important part of the recommended daily allowances of vitamins and minerals and any deficiency therein can lead to health risks such as osteoporosis, hypertension, and/or weak bone density.

Young children who have lactose intolerance are very rare. The amount of lactase enzyme a body produces generally reaches a maximum immediately after birth and then decreases in the majority of people after their body adjusts during the ages of about 3-15.

Generally, humans develop lactose intolerance from a primary or secondary cause. The primary cause is an onset of loss of lactase that is believed to be a permanent condition. This occurs at a variable period after the weaning period. The primary cause is also genetically determined. The secondary cause is generally a temporary condition that occurs as a result of another disease or event that damages the lining of the small intestine where lactase is active. This is usually caused by an acute diarrhea, disease, parasitic infection, Crohn's disease, celiac disease, gastrointestinal surgery, or the intake of certain medications.

In addition to the primary and secondary causes, certain human ethnic and racial populations have more of a predisposition for lactose intolerance. In these populations, social and cultural habits and attitudes influence lactose intolerance. Lactose activity can also decrease with age in certain ethnic and racial populations, including those populations which have origins in Europe, the African plains, and the Siberian Steppes. Humans who are most likely to have or develop lactose intolerance include those of Asian, Middle Eastern, North American, African, and Latin American decent.

Previous attempts at improving the symptoms of lactose intolerance have been met with some success, e.g., U.S. Pat. No. 7,029,702.

### SUMMARY OF THE INVENTION

Described herein is a composition comprising a lactose composition with decreased lactose content and an effective amount of a probiotic, a prebiotic, or a mixture thereof. In one aspect, a composition is provided comprising a lactose composition with decreased lactose content and an effective amount of a probiotic, a prebiotic, or a mixture thereof, wherein the lactose composition with decreased lactose content comprises at least about 0.001% lactose. In one embodiment, the lactose composition with decreased lactose content comprises lactose present in a substance chosen from the group consisting of milk or milk products comprising flavored-milk, yogurt, a yogurt drink, a cheese, butter, ice cream, sherbet, a liquado, and a smoothie. In another embodiment, the lactose composition with decreased lactose content comprises non-fat, reduced-fat, or whole-fat compositions. In another embodiment, the lactose composition with decreased lactose content comprises about 0.01% to about 5.3% lactose. In another embodiment, the lactose composition with decreased lactose content comprises about 0.01% to about 2.7% lactose. In another embodiment, the lactose composition with decreased lactose content comprises about 0.1% to about 5.3% lactose. In another embodiment, the lactose composition with decreased lactose content comprises about 0.1% to about 2.7% lactose.

In one embodiment, the prebiotic comprises a carbohydrate polymer. In one embodiment, the prebiotic comprises one or more of a fructo-oligosaccharide (FOS), a galacto-oligosaccharide (GOS), a transgalacto-oligosaccharide (TOS), or a xylo-oliogosaccharide (XOS). In another embodiment, the GOS and/or TOS comprise β(1-4) linkages, β(1-6) linkages, or a combination of both. In another embodiment, the carbohydrate polymer comprises about 0.1 g to about 15 g per 240 g serving. In another embodiment, the prebiotic is lactulose.

In one embodiment, the probiotic comprises a member of the genera lactobacillus, bifidobacteria, or mixtures thereof. In another embodiment, the probiotic comprises about 1×10⁶ cfu's to about 1×10⁹ cfu's per 240 g serving. In another embodiment, the probiotic comprises about 0.001 mg to about 1 mg per 240 g serving.

In another aspect, a method is provided comprising providing to a subject a composition comprising a lactose composition with decreased lactose content and an effective amount of a probiotic, a prebiotic, or a mixture thereof, wherein the lactose composition with decreased lactose content comprises at least about 0.001% lactose. In one embodiment, the lactose composition with decreased lactose content comprises lactose present in a substance chosen from the group consisting of milk or milk products comprising flavored-milk, yogurt, a yogurt drink, a cheese, butter, ice cream, sherbet, a liquado, and a smoothie. In one embodiment, the lactose composition with decreased lactose content comprises non-fat, reduced-fat, or whole-fat compositions.

In one embodiment, the lactose composition with decreased lactose content comprises at least 0.01% lactose. In one embodiment, the lactose composition with decreased lactose content comprises at least 0.1% lactose. In one embodiment, the lactose composition with decreased lactose content comprises about 0.01% to about 5.3% lactose. In one embodiment, the lactose composition with decreased lactose content comprises about 0.1% lactose to about 5.3% lactose. In one embodiment, the lactose composition with decreased lactose content comprises about 0.01% to about 2.7% lactose. In one embodiment, the lactose composition with decreased lactose content comprises about 0.1% to about 2.7% lactose. In one embodiment, the lactose composition with decreased lactose content comprises about 0.01% to about 7% lactose. In one embodiment, the lactose composition with decreased lactose content comprises about 0.1% to about 7% lactose. In one embodiment, the lactose composition with decreased lactose content comprises about 0.01% lactose to about 25% lactose. In one embodiment, the lactose composition with decreased lactose content comprises about 0.1% lactose to about 25% lactose.

In one embodiment, the prebiotic comprises a carbohydrate polymer. In one embodiment, the prebiotic comprises one or more of a fructo-oligosaccharide (FOS), a galacto-oligosaccharide (GOS), a transgalacto-oligosaccharide (TOS), a xylo-oliogosaccharide (XOS). In one embodiment, the GOS and/or TOS comprise β(1-4) linkages, β(1-6) linkages, or a combination of both. In one embodiment, said prebiotic is lactulose. In one embodiment, the probiotic comprises a member of the genera lactobacillus, bifidobacteria, or mixtures thereof. In one embodiment, the probiotic comprises about 1×10⁶ cfu's to about 1×10⁹ cfu's per 240 g serving. In one embodiment, the probiotic comprises about 0.001 mg to about 1 mg per 240 g serving.

### INCORPORATION BY REFERENCE

All publications, patents, and patent applications mentioned in this specification are herein incorporated by reference in their entirety to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates that lactose exists as alpha- and beta-lactose.

FIG. 2 illustrates examples of GOS.

FIG. 3 illustrates lactulose.

### DETAILED DESCRIPTION OF THE INVENTION

The invention provides methods and compositions useful for the reduction of symptoms of lactose intolerance and for improving overall gastrointestinal (GI) health. Symptoms of lactose intolerance include gas, bloating, flatulence, diarrhea, abdominal pain, cramping, and vomiting. Minor digestive problems related to the GI also include occasional bloating, diarrhea, constipation, gas, heartburn, or stomach upset. The methods and compositions described herein can be useful for reducing or eliminating one or more of these symptoms, for example through colonic adaptation. The invention relates to lactose compositions with decreased lactose content comprising one or more prebiotics and/or one or more probiotics. These compositions are expected to modify the colonic flora, which can result in an increased tolerance to lactose and other fermentable carbohydrates. Furthermore, these compositions allow the colonic flora, comprising microorganisms known to increase the ability of an individual to tolerate fermentable carbohydrates, to be regularly replenished through consumption of the compositions. Adaptation of the colonic flora allows the colon's capacity to handle gas to be used for other challenges. By improving the composition of the colonic flora, the colon's capacity to handle compositions without decreased lactose amounts is increased. For example, an individual's tolerance to dairy in general can be improved through regular consumption of these lactose compositions with decreased lactose content. This change in colonic flora is useful for the reduction of bloating, diarrhea, gastric distention and pain, and/or flatulence from the consumption of dairy products and other lactose compositions. Herein, “lactose composition with decreased lactose content” refers to a composition with decreased lactose amounts as compared to the corresponding composition with normal amounts of lactose. In some embodiments, the compositions with decreased lactose amounts are dairy compositions.

### Testing Lactose Intolerance

Lactose intolerance can be tested either indirectly or directly. There are three main ways to test by the indirect method: a hydrogen breath test, a stool acidity test, or a blood glucose test. In the hydrogen breath test, breath is measured to determine the amount of hydrogen produced after consuming a measured amount of lactose, e.g., 15 g. Lactose is consumed by a subject by drinking a lactose mixture, and the subject exhales into a vacuum-sealed collection tube at three one hour time intervals. A high level of hydrogen in the breath can indicate an improper digestion of lactose. In a stool test, the stool is tested to determine the amount of acid. In a blood glucose test, blood is tested to determine the amount of glucose (sugar) content after administering a predetermined amount of lactose-containing product to the subject. The direct method measures lactase activity in a mucosal biopsy specimen.

The stool acidity test is typically used to test lactose intolerance in infants and young children. The hydrogen breath test is typically not recommended for young children since dehydration can occur due to diarrhea after ingestion of the lactose-containing drink.

### Types of Lactose Intolerance and Treatments

People can have different degrees of lactose intolerance. Lactose intolerance can be psychologically induced. There are many different variations of lactose intolerance depending on the individual. For example, some individuals cannot digest cheese, melted cheese, plain milk, or warm dairy containing products like milk in coffee, while others cannot digest any dairy products at all. Also, most lactose intolerant people are limited as to the amount of special “lactose free” foods they can eat, without displaying symptom of lactose intolerance, that have been manufactured by specified companies. Some examples of these “lactose free” foods are: MOCHA MIX® ice cream, TOFUTTI® ice cream and ice cream sandwiches, LACTAID® brand milk, FORMAGG™ cheese, TOFUTTI® “Better than Cream Cheese”, margarine, and live cultured yogurt.

Use of lactase tablets can help lactose intolerant people digest milk and milk products. Each lactase tablet can hydrolyze up to 99% of the ingested lactose within 24 hours and is designed to be ingested with the lactose containing food. Still other techniques for dealing with lactose maldigestion are to use microgranules containing bioactive compounds or microorganisms (see, e.g., U.S. Pat. No. 5,952,021). The use of an active lactase composition for treatment of lactase deficiency is described in U.S. Pat. No. 3,718,739.

Lactose Compositions with Decreased Lactose Content

Lactose compositions with decreased lactose content described herein can include dairy products such as fluid milk or milk-products. Milk products can be obtained, for example, from cows, buffalos, sheep, or goats. Examples can include, but are not limited to, milk (whole, reduced-fat, skim, semi-skim, flavored, buttermilk, etc.), yogurt, yogurt drinks, cheese, butter, margarine, oil-based spreads, creamers, half and half, or the like. Non-limiting examples of cheeses that can be used as the lactose compositions with decreased lactose content include cottage cheese, cheddar cheese, ricotta cheese, cream cheese, other cheese products or the like. Lactose compositions with decreased lactose content can also comprise ice cream, gelato, frozen yogurt, sherbet, shakes, malts, smoothies, liquados, or other similar products. Other examples can include fermented products such as fermented milk products, sour cream, creme fraiche, and the like. Reduced-fat or non-fat versions of the above examples can also be used as the lactose composition with decreased lactose content.

A lactose composition with decreased lactose content can also comprise various flavors including, but not limited to, vanilla, strawberry, raspberry, mixed berry, prune, peach, blueberry, cherry, lemon, or chocolate. The compositions can also be un-flavored or plain flavored.

The compositions of the provided invention can contain lactose in a range of concentrations. All percentage values refer to weight of lactose to weight of total composition, unless otherwise specified. In some embodiments, the minimum lactose amounts can be at least about 0.001%, about 0.01%, about 0.1%, about 1%, about 2%, about 3%, about 4%, about 5%, about 7.5%, about 10%, about 15%, or about 20% of the composition. Other minimum amounts of lactose are suitable as long as the lactose amount has been decreased compared to those amounts in the corresponding lactose composition without decreased lactose content. Maximum amounts of lactose can be any amounts which are decreased compared to those amounts in the corresponding lactose composition without decreased lactose content. In some embodiments, the maximum lactose amounts can be about 0.001%, about 0.01%, about 0.1%, about 1%, about 2%, about 2.7%, about 5.3%, about 6%, about 7%, about 10%, about 15%, about 20%, or about 25% of the composition.

Ranges of suitable lactose amounts in the lactose compositions with decreased lactose content can be from about 0.001% to about 2.7%, about 0.001% to about 5.3%, about 0.001% to about 6%, about 0.001% to about 7%, about 0.001% to about 10%, about 0.001% to about 15%, about 0.001% to about 20%, about 0.001% to about 25%. In some embodiments, suitable ranges can also be from about 0.01% to about 2.7%, about 0.01% to about 5.3%, about 0.01% to about 6%, about 0.01% to about 7%, about 0.01% to about 10%, about 0.01% to about 15%, about 0.01% to about 20%, about 0.01% to about 25%. In other embodiments, suitable ranges can also be from about about 0.1% to about 2.7%, about 0.1% to about 5.3%, about 0.1% to about 6%, about 0.1% to about 7%, about 0.1% to about 10%, about 0.1% to about 15%, about 0.1% to about 20%, about 0.1% to about 25%. Other suitable ranges can also be from about about 1% to about 2.7%, about 1% to about 5.3%, about 1% to about 6%, about 1% to about 7%, about 1% to about 10%, about 1% to about 15%, about 1% to about 20%, and about 1% to about 25%.

In some embodiments, the minimum lactose amounts can be at least about 0.002 g, about 0.02 g, about 0.2 g, about 2 g, about 5 g, about 10 g, about 15 g, about 20 g, about 25 g, about 30 g, about 35 g, about 40 g, about 45 g, about 50 g, about 55 g, about 60 g, of the serving. Other minimum amounts of lactose are suitable as long as the lactose amount has been decreased compared to those amounts in the corresponding lactose composition without decreased lactose contents. Maximum amounts of lactose can be any amounts which are decreased compared to those amounts in the corresponding lactose composition without decreased lactose contents. In some embodiments, the maximum lactose amounts can be about, 0.002 g, about 0.02 g, about 0.2 g, about 2 g, about 5 g, about 10 g, about 15 g, about 20 g, about 25 g, about 30 g, about 35 g, about 40 g, about 45 g, about 50 g, about 55 g, about 60 g, of the serving.

Ranges of suitable lactose amounts in the lactose compositions with decreased lactose content can be from about 0.002 g to about 0.5 g, about 0.002 g to about 1 g, about 0.002 g to about 2 g, about 0.002 g to about 5 g, about 0.002 g to about 8 g, about 0.002 g to about 10 g, about 0.002 g to about 15 g, about 0.002 g to about 20 g, about 0.002 g to about 25 g, about 0.002 g to about 30 g, about 0.002 g to about 40 g, about 0.002 g to about 50 g, about 0.002 g to about 60 g. In some embodiments, suitable ranges can also be from about 0.02 g to about 0.5 g, about 0.02 g to about 1 g, about 0.02 g to about 2 g, about 0.02 g to about 5 g, about 0.02 g to about 8 g, about 0.02 g to about 10 g, about 0.02 g to about 15 g, about 0.02 g to about 20 g, about 0.02 g to about 25 g, about 0.02 g to about 30 g, about 0.02 g to about 40 g, about 0.02 g to about 50 g, about 0.02 g to about 60 g. In other embodiments, suitable ranges can also be from about 0.2 g to about 0.5 g, about 0.2 g to about 1 g, about 0.2 g to about 2 g, about 0.2 g to about 5 g, about 0.2 g to about 8 g, about 0.2 g to about 10 g, about 0.2 g to about 15 g, about 0.2 g to about 20 g, about 0.2 g to about 25 g, about 0.2 g to about 30 g, about 0.2 g to about 40 g, about 0.2 g to about 50 g, about 0.2 g to about 60 g. Other suitable ranges can also be from about 1 g to about 2 g, about 1 g to about 5 g, about 1 g to about 8 g, about 1 g to about 10 g, about 1 g to about 15 g, about 1 g to about 20 g, about 1 g to about 25 g, about 01 g to about 30 g, about 1 g to about 40 g, about 1 g to about 50 g, about 1 g to about 60 g

Depending on the lactose composition with decreased lactose content, serving sizes can vary. For example, a serving can be, but not limited to, a cup, an ounce, a pat, a tbsp, or half a cup. Due to density differences among various dairy products, it is understood that the weight per serving size can need adjustment for determining the percentage of lactose in the serving size of a particular composition. For example, fluid milk and milk products can have a serving size of about 240 g, or about 245 g, or about 240 g to about 245 g, or about 227 g to about 300 g. Yogurt can have a serving size of about 4 oz, or about 6 oz, or about 8 oz, or about 4 oz to 10 oz, or about half cup, or about 1 cup, or about 113 g, or about 170 g, or about 227 g, or about 245 g or about 277 g, or about 100 g to about 350 g.

Methods to produce the lactose composition with decreased lactose content are known to those skilled in the art. For example, addition of lactase can be used to treat regular milk to hydrolyze the lactose into glucose and galactose. Some examples of methods to produce milk with decreased lactose content are described in U.S. Pat. Nos. 5,707,843; 5,357,852; 6,881,428; 4,957,752; and 4,956,186. Ultrafiltration can also be useful for producing lactose compositions with decreased lactose content. In other embodiments, polymerization of lactose using enzymes can be useful for generating carbohydrate polymers and can therefore result in decreased lactose content (see, e.g., U.S. Pat. Nos. 5,952,205 and 6,423,833).

The lactose compositions can have completely decreased lactose amounts, i.e., near or less than 100%. In an embodiment, the lactose composition with decreased lactose content can have lactose decreased from a range of about 0.5% to about 99.99%, compared to the composition without decreased lactose amounts. In other embodiments, the level of decreased lactose amounts can be about 0.5%, about 1%, about 5%, about 10%, about 20%, about 30%, about 40%, about 50%, about 60%, about 70%, about 80%, about 90%, about 95%, about 98%, or about 99.9%. The level of decreased lactose amounts can also be from about 1% to about 99.99%, about 10% to about 99.99%, about 20% to about 99.99%, about 30% to about 99.99%, about 40% to about 99.99%, about 50% to about 99.99%, about 60% to about 99.99%, about 70% to about 99.99%, about 80% to about 99.99%, about 90% to about 99.99%, about 98% to about 99.99%, or about 99% to about 99.99%. The level of decreased lactose amounts can also be from about 1% to about 90%, about 10% to about 90%, about 20% to about 90%, about 30% to about 90%, about 40% to about 90%, about 50% to about 90%, about 60% to about 90%, about 70% to about 90%, about 80% to about 90%, or about 85% to about 90%. In some embodiments, the level of decreased lactose amounts can also be from about 1% to about 80%, about 10% to about 80%, about 20% to about 80%, about 30% to about 80%, about 40% to about 80%, about 50% to about 80%, about 60% to about 80%, about 70% to about 80%, or about 75% to about 80%. In other embodiments, the level of decreased lactose amounts can also be from about 1% to about 50%, about 10% to about 50%, about 20% to about 50%, about 30% to about 50%, about 40% to about 50%, or about 45% to about 50%.

Another way of specifying the level of decreased lactose in a lactose composition is in weight per serving. The lactose composition with decreased lactose amounts can have lactose reduced by about 1 g to about 60 g, compared to the composition without decreased lactose amounts. In other embodiments, the lactose amounts can be decreased by about 50 g, 40 g, about 30 g, about 20 g, about 10 g, about 5 g, about 1 g, or about 0.5 g. The lactose amounts can be decreased from about 1 g to about 60 g, about 10 g to about 60 g, about 20 g to about 60 g, about 30 g to about 60 g, about 40 g to about 60 g, about 50 g to about 60 g, or about 55 g to about 60 g. The lactose amounts can be decreased from about 1 g to about 50 g, about 10 g to about 50 g, about 20 g to about 50 g, about 30 g to about 50 g, or about 40 g to about 50 g. The lactose amounts can be decreased from about 1 g to about 50 g, about 10 g to about 50 g, about 20 g to about 50 g, about 30 g to about 50 g, or about 40 g to about 50 g. The lactose amounts can be decreased from about 1 g to about 40 g, about 10 g to about 40 g, about 20 g to about 40 g, about 30 g to about 40 g. The lactose amounts can be decreased from about 1 g to about 20 g, about 5 g to about 20 g, about 10 g to about 20 g, or about 15 g to about 20 g. The lactose amounts can be decreased from about 1 g to about 100 g, about 2 g to about 10 g, about 3 g to about 10 g, about 4 g to about 10 g, about 5 g to about 10 g, about 6 g to about 10 g, about 7 g to about 10 g, about 8 g to about 10 g, or about 9 g to about 10 g. The lactose amounts can be decreased from about 1 g to about 5 g, about 2 g to about 5 g, about 3 g to about 5 g, or about 4 g to about 5 g.

Lactose compositions with decreased lactose content can contain one or more probiotics and/or one or more prebiotics.

### Probiotics

In some embodiments, the lactose compositions with reduced lactose content comprise one or more probiotics and/or one or more prebiotics. Probiotics (or probiotic bacteria) typically refer to beneficial live microorganisms, e.g., bacteria, found in the gastrointestinal tract and, when administered in adequate amounts, confer a health benefit on the host (or subject in need thereof) such as helping to maintain a healthy immune system, or increasing the ability of the colon to slow the rate of fermentation. Probiotics favorably alter the intestinal flora balance, inhibit the growth of harmful bacteria, promote good digestion, boost immune function, and increase resistance to infection. People with flourishing intestinal colonies of beneficial bacteria are better equipped to fight the growth of disease-causing bacteria. Any suitable bacteria for assisting in reduction or elimination of lactose intolerance-like symptoms or improving overall GI health, for example through colonic adaptation, can be used in the methods and compositions described herein.

Examples of probiotics include, but are not limited to, those that acidify the colon such as those from the genera Lactobacillus or Bifidobacteria, which are thought to maintain a healthy balance of intestinal flora by producing organic compounds, such as lactic acid, hydrogen peroxide, and acetic acid, resulting in increased acidity of the intestine and inhibiting the reproduction of many harmful bacteria. Probiotics also produce substances called bacteriocins, which act as natural antibiotics to help eliminate undesirable microorganisms.

Non-exclusive examples of probiotic bacteria that can be used in the methods and compositions described herein include Lactobacillus acidophilus or L. acidophilus. Acidophilus, a probiotic, is a strain of the Lactobacilli family of gut flora which inhabit the GI tract. These beneficial bacteria are involved with immune system function, inhibiting carcinogenesis, metabolism of cholesterol, aging, and nutritional status. Acidophilus and other probiotics help maintain optimum pH, reduce putrefaction, and reduce endotoxemia. Other Lactobacillus bacteria which can be employed include, but are not limited to, L. crispatus, L. casei, L. rhamnosus, L. reuteri, L. fermentum, L. plantarum, L. sporogenes, and L. bulgaricus.

Other probiotic bacteria suitable for the compositions include Bifidobacterium lactis, B. animalis, B. bifidum, and B. infantis. Yeasts, such as Saccharomyces boulardii, are also suitable as probiotics and can act to restore the intestinal flora. Mixtures of one or more species or strains of bacteria can be used. For example, yogurt already contains the bacteria species Lactobacillus bulgaricus and Streptococcus thermophilus used for fermentation and can contain additional species of probiotics and can also be supplemented with prebiotics. Other probiotic bacteria suitable for use in the lactose compositions with reduced lactose content of the provided invention include Bacillus coagulans GBI-30, 6086; Bifidobacterium animalis subsp. lactis BB-12; Bifidobacterium breve Yakult; Bifidobacterium infantis 35624; Bifidobacterium animalis subsp. lactis HN019 (DR10); Bifidobacterium longum BB536; Escherichia coli M-17; Escherichia coli Nissle 191; Lactobacillus acidophilus DDS-1; Lactobacillus acidophilus LA-5; Lactobacillus acidophilus NCFM; Lactobacillus casei DN114-001 (Lactobacillus casei Immunitas(s)/Defensis); Lactobacillus casei CRL431; Lactobacillus casei F19; Lactobacillus casei Shirota; Lactobacillus paracasei Stl 1 (or NCC2461); Lactobacillus johnsonii La1 (=Lactobacillus LC1); Lactococcus lactis L1A; Lactobacillus plantarum 299V; Lactobacillus reuteri ATTC 55730 (Lactobacillus reuteri SD2112); Lactobacillus rhamnosus ATCC 53013; Lactobacillus rhamnosus LB21; Saccharomyces boulardii lyo; mixture of Lactobacillus rhamnosus GR-1 & Lactobacillus reuteri RC-14; mixture of Lactobacillus acidophilus NCFM & Bifidobacterium bifidum BB-12; Lactobacillus acidophilus CL1285 & Lactobacillus casei; Lactobacillus helveticus R0052 & Lactobacillus rhamnosus R0011.

Any suitable amount of probiotic per serving can be used. The dose can be about 0.001 mg to about 1 mg, or about 0.5 mg to about 5 mg, or about 1 mg to about 1000 mg, or about 2 to about 200 mg, or about 2 to about 100 mg, or about 2 to about 50 mg, or about 4 to about 25 mg, or about 5 to about 20 mg, or about 10 to about 15 mg, or about 50 mg to about 200 mg, or about 200 mg to about 1000 mg or about 10, 11, 12, 12.5, 13, 14, or 15 mg per serving. In some embodiments, L. acidophilus is used in a dose of about 12.5 mg per serving. The probiotic can also be about 0.5 w/w to about 20% w/w of the final lactose-reduced dairy composition. A dose of probiotic can be given in combination with one or more prebiotics, which are further described herein.

Another common way of specifying the amount of probiotics is as a colony forming unit (cfu). A cfu is an individual cell which is able to clone itself into an entire colony of identical cells. In some embodiments, one or more strains of probiotic bacteria are ingested in an amount of about 1×10⁶ to about 1×10⁹ cfu's, or about 1×10⁶ cfu's to about 1×10⁹ cfu's, or about 10×10⁶ cfu's to about 0.5×10⁹ cfu's, or about 113×10⁵ cfu's to about 113×10⁶ cfu's, or about 240×10⁵ cfu's to about 240×10⁶ cfu's or about 0.3×10⁹ cfu's per serving. In another embodiment, one or more strains of probiotic bacteria are administered as part of a lactose composition with decreased lactose content. In some embodiments, a typical serving size for a dairy product such as fluid milk is about 240 g. In other embodiments, a serving size is about 245 g, or about 240 g to about 245 g, or about 227 to about 300 g. In one embodiment the dairy product is yogurt with a decreased lactose content. Yogurt can have a serving size of about 4 oz, or about 6 oz, or about 8 oz, or about 4 oz to 10 oz, or about half cup, or about 1 cup, or about 113 g, or about 170 g, or about 227 g, or about 245 g or about 277 g, or about 100 g to about 350 g. In one embodiment, probiotic bacteria, e.g. L. acidophilus are provided in a lactose composition with decreased lactose content.

In one embodiment probiotic bacteria are provided as live cultured bacteria, e.g. in combination with a prebiotic (comprising or consisting essentially of GOS, TOS, GOS and TOS, lactulose, or XOS) in a lactose composition with decreased lactose content. The dose of probiotic bacteria can be about 1 to about 1000 mg, or about 2 to about 200 mg, or about 2 to about 100 mg, or about 2 to about 50 mg, or about 4 to about 25 mg, or about 5 to about 20 mg, or about 10 to about 15 mg, or about 10, 11, 12, 12.5, 13, 14, or 15 mg. In one embodiment, L. acidophilus is used in a dose of about 12.5 mg.

In one embodiment, an effective amount of freeze dried probiotic bacteria can be introduced into a lactose composition with decreased lactose content.

### Prebiotics

A prebiotic is generally a carbohydrate (saccharide) that is indigestable or essentially indigestable by a human and can act to encourage the growth of probiotic bacteria in the gut that alleviate symptoms of lactose intolerance, increase adhesion of probiotic bacteria in the gut, or allow doses of probiotic bacteria to more readily pass through the stomach without being destroyed. Prebiotics contain saccharide parts that are indigestible and can act as a non-digestible fiber in the diet. This is because humans lack the enzymes to break down some parts of the prebiotic as it travels down the digestive tract. When the prebiotic reaches the large intestine and the colon, the probiotic bacteria that are found there start to break down the prebiotics since the probiotics have the enzymes needed to break down the prebiotics. For instance, Bifidobacteria have been reported to digest prebiotic saccharides. It is generally believed that foods that promote Bifidobacteria growth are good for the health.

Prebiotics suitable for a lactose composition with decreased lactose content can include a carbohydrate, carbohydrate monomer, carbohydrate oligomer, or carbohydrate polymer. In one embodiment, the prebiotic is an indigestible saccharide, which includes indigestible monosaccharides, indigestible oligosaccharides, or indigestible polysaccharides. In one embodiment, the sugar units of a oligosaccharide or polysaccharide can be linked in a single straight chain or can be a chain with side branches. The length of the oligosaccharide or polysaccharide can vary from source to source. In some embodiments, glucose can also be contained in the chain. In other embodiments, the prebiotics can be partially hydrolyzed.

Examples of prebiotics suitable for a lactose composition with decreased lactose content include, but are not limited to, galacto-oligosaccharide (GOS), raffinose, stachyose, lactose, fructans, galactan, food gum, mannan-oligosaccharide (MOS), fructo-oligosaccharide (fructose polymers; FOS; i.e. oliogfructose or oligofructan), psyllium, lactulose, guar, gellan, konjac, neosugar, carrageenan, inulin (an example of a longer chained FOS), fructo-inulins, lactitol, lactosucrose, oligofructose, pyrodextrins, soybean oligosaccharides (i.e. soy oligosaccharides), transgalactosylated oligosaccharides (i.e. transgalacto-oligosaccharides (TOS)), transgalactosylate disaccharides, gentioologosaccharides, glucooligosaccharides, pecticoligosaccharides, palatinose polycondensates, difructose anhydride III, sorbitol, maltitol, lactitol, polyols, polydextrose, reduced paratinose, cellulose, β-glucose, β-galactose, β-fructose, verbascose, galactinol, β-glucan, guar gum, pectin, sodium alginate, lambda carrageenan, xylo-oliogosaccharides (XOS), paratinose oligosaccharide, or mixtures thereof.

Prebiotics can promote colonic bacteria that slow fermentation. For example, fructo-oliogosaccharides (FOS), neosugar, or inulin promote the growth of acid forming bacteria in the colon such as bacteria belonging to the genera Lactobacillus or Bifidobacteria. For instance, L. acidophilus and B. bifidus play a role in reducing the number of pathogenic bacteria. Additional nutritional properties, such as the effect on colonic pH and stool bulking justify their classification as dietary fibers. In experimental models, it has also been reported that they improve the bioavailability of essential minerals. As a fiber, it is thought to slow digestion and allow the painless reintroduction of lactose into the body. Other polymers, such as various galactans, and carbohydrate based gums, such as psyllium, guar, carrageen, gellan, konjac are also known to improve GI health. The carbohydrate lactulose is also known to improve GI gas handling capacity.

### Oligosaccharides

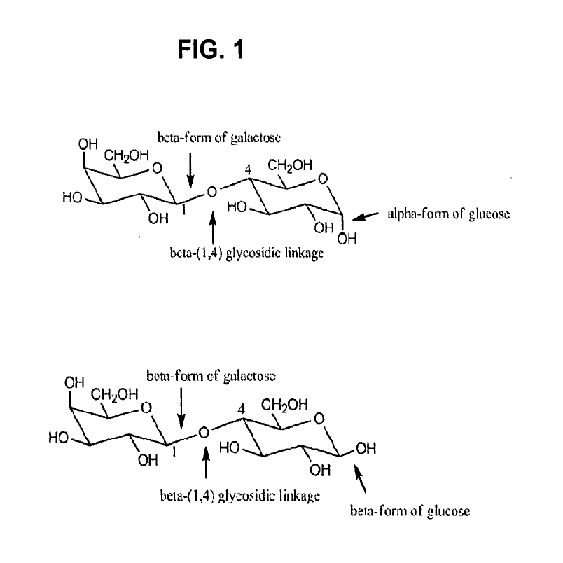

Oligosaccharides are generally considered to have a reducing end and a non-reducing end, whether or not the saccharide at the reducing end is in fact a reducing sugar. In accordance with accepted nomenclature, most oligosaccharides are depicted herein with the non-reducing end on the left and the reducing end on the right. Most oligosaccharides described herein are described with the name or abbreviation for the non-reducing saccharide (e.g. Gal or D-Gal), preceded or followed by the configuration of the glycosidic bond (α or β), the ring bond, the ring position of the reducing saccharide involved in the bond, and then the name or abbreviation of the reducing saccharide (e.g. Glc or D-Glc). The linkage (e.g. glycosidic linkage, galactosidic linkage, glucosidic linkage) between two sugar units can be expressed, for example, as 1,6, 1-->6, or (1-6). Each saccharide is in the cyclic form (pyranose or furanose form). For example, lactose is a dissaccharide composed of cyclic forms of galactose and glucose joined by a β(1-4) linkage where the acetal oxygen bridge is in the beta orientation. Lactose can exist as α- and β-lactose (see FIG. 1). β-lactose can be expressed as β-D-galactopyranosyl-(1-4)β-D-glucopyranose, β-D-Gal-(1-4)-β-D-Glc or as Gal β(1-4)-Glc. α-lactose can be expressed as β-D-galactopyranosyl-(1-4) α-D-glucopyranose, β-D-Gal-(1-4)-α-D-Glc or as Gal β(1-4)-Glc.

Galacto-oligosaccharides (GOS) are oligomers or polymers of galactose molecules ending mainly with a glucose or sometimes ending with a galactose molecule and have varying degree of polymerization (DP=2-20) and type of linkages. In one embodiment, GOS comprises galactose and glucose molecules. In another embodiment, GOS comprises only galactose molecules. In a further embodiment, galacto-oligosaccharides (GOS) are galactose-containing oligosaccharides of the form of [β-D-Gal-(1-6)]ₙ-β-D-Gal-(1-4)-D-Glc wherein n is 0-10. In another embodiment, GOS are galactose-containing oligosaccharides of the form Glc α1-4-[βGal 1-6]ₙ where n=2-8. In another embodiment, GOS are in the form of α-D-Glc (1-4)-[β-D-Gal-(1-6)-]ₙ where n=1-7. Gal is a galactopyranose unit and Glc (or Glu) is a glucopyranose unit.

GOS is found in human and bovine maternal milk. GOS can also be produced from lactose syrup using the transgalactosylase activity of the enzyme β-galactosidase (Crittenden, (1999) Probiotics: A Critical Review. Tannock, G. (ed) Horizon Scientific Press, Wymondham, pp. 141-156). β-D-galactosidase is known to catalyze not only the hydrolysis of the 3-D-galactoside linkage of lactose to give D-glucose and D-galactose but also to carry out transgalactosylation reactions where the D-galactosyl group of a β-D-galactoside is transferred onto a hydroxylated acceptor. For example, when a β-D-galactoside such as lactose or another carbohydrate is present, it is possible to obtain new glycoside linkages between the D-galactose unit and the acceptor. The starting galactoside such as lactose can also be present in the GOS mixture following the transgalactosylation reactions. As used herein, GOS comprises one or more saccharides that have been produced from a glycoside and the transgalactosylation reaction of a β-galactosidase. Thus, GOS includes saccharides such as transgalactosylated oligosaccharides (i.e. transgalacto-oligosaccharides) or transgalactosylate disaccharides. The DP of the formed oligosaccharide can vary, typically from 2-20, depending on the enzyme source. In one embodiment, GOS is a blend of one more saccharides with a DP range of 2-6 (i.e. di-through hexasaccharides). In another embodiment, GOS is a blend of one or more saccharides with a DP range of 2-8 (i.e. di- through octasaccharides). In another embodiment, GOS is a blend of one or more saccharides with a DP range of greater than 8. In yet another embodiment, GOS is a blend of one or more saccharides with a DP range of 9-15. In another embodiment, GOS is a blend of one or more saccharides with a DP of 1, a DP range of 2-6, a DP range of 6-8, and DP range of greater than 8.

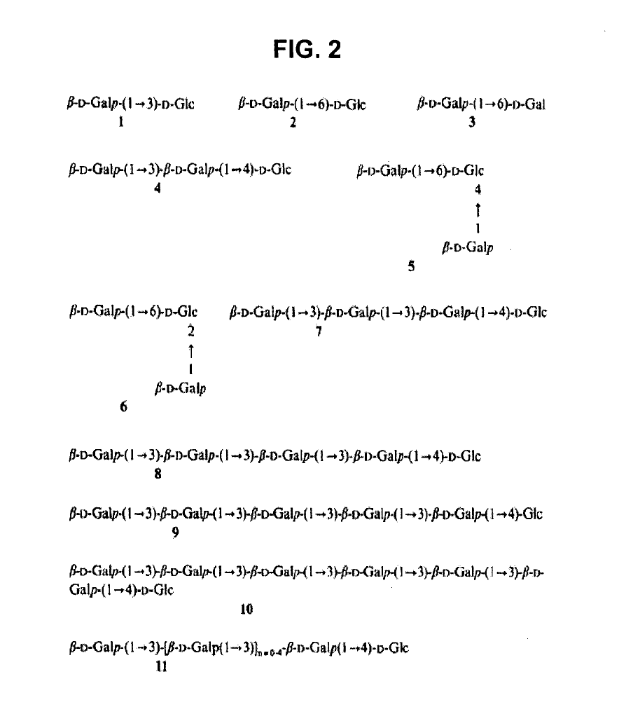

Linkages between the individual sugar units found in GOS include β-(1-6), β-(1-4), β-(1-3) and β-(1-2) linkages. β-(1-3) linkages are less common than β-(1-6), β-(1-4) linkages. Linkages between individual sugars in TOS include β-(1-6) and β-(1-4). In one embodiment, GOS comprises a number of β-(1-6) linked or β-(1-4) galactopyranosyl units linked to a terminal glucopyranosyl residue through an α-(1-4) glycosidic bond. In another embodiment, GOS comprises a number of β-(1-6) linked or β-(1-4) galactopyranosyl units linked to a terminal glucopyranosyl residue through an β-(1-4) glycosidic bond. In another embodiment, GOS formed by transgalactosylation comprise β-D-galactopyranosyl-(1-3) linkages. In one embodiment, GOS are branched saccharides. Branched oligosaccharides can be formed as an artifact of the transgalactosylation reaction. In another embodiment, GOS are linear saccharides. Non-limiting GOS examples include those in FIG. 2.

The source of the β-galactosidase can determine the GOS end products from a transgalactosylation reaction. For example, β-galactosidase from Streptococcus thermophilus can produce a collection of transgalactosylated disaccharides including Galβ (1-6) Glc, Galβ (1-3) Glc, Galβ (1-2) Glc, and Galβ (1-6) Gal (Matsumoto et al., (1992), Chapter 5: Galactooligosaccharides, in Japanese Technology Reviews, ed. By Karbe, I., Gordon and Breach, NY, pp. 90-160). Transgalactosylated oligosaccharides (TOS) can be produced using (β-galactosidase from Aspergillus oryzae (Tanaka et al, (1983) Bifidobacteria Microflora, 2, 17-24), and consist of tri-, tetra-, penta- and hexa-galactooligosaccharides. Another type of GOS is Oligomate 55, which is prepared using β-galactosidase from A. oryzae and Streptococcus thermophilus (Ito et al., (1990), Microbial Ecology in Health and Disease, 3, 285-292) and contains 36% tri-, tetra-, penta- and hexa-galactooligosaccharides, 16% disaccharides galactosyl-glucose and galactosyl-galactose, 38% monosaccharides, and 10% lactose.

Alpha-GOS (also called alpha-bond GOS or alpha-linked GOS) are oligosaccharides having an alpha-galactopyranosyl group. Alpha-GOS comprises at least one alpha glycosidic linkage between the saccharide units. Alpha-GOS are generally represented by α-(Gal)ₙ (n usually represents an integer of 2 to 10) or α-(Gal)ₙ Glc (n usually represents an integer of 1 to 9). Examples include a mixture of α-galactosylglucose, α-galactobiose, α-galactotriose, α-galactotetraose, and higher oligosaccharides. Additional non-limiting examples include melibiose, manninootriose, raffinose, stachyose and the like, which can be produced from beat, soy bean oligosaccharide and the like.

Commercially available and enzyme synthesized alpha-GOS products are also useful for the compositions described herein. Synthesis of alpha-GOS with an enzyme is conducted utilizing the dehydration condensation reaction of α-galactosidase with the use of galactose, galactose-containing substance, or glucose as a substrate. The galactose-containing substance includes hydrolysates of galactose-containing substances, for example, a mixture of galactose and glucose obtained by allowing beta-galactosidase and acid to act on lactose, and the like. Glucose can be mixed separately with galactose and used as a substrate with α-galactosidase (see e.g. PCT Publication WO 02/18614). Methods of preparing alpha-GOS have been described (see e.g. EP1514551 and EP2027863).

FOS are chain oligomers or polymers of the sugar fructose that can be found in a variety of foods. The sugar units can be linked in a single straight chain or can be a chain with side branches. In many cases small amounts of glucose are also contained in the chain. The length of the fructose chains can vary from source to source. FOS are primarily polyfructans with a degree of polymerization (DP) generally ranging from 2 to 20 (oligofructose) or greater than 20 (inulin). Generally, the D-fructose moieties in FOS are joined by β-(2-1) linkages and the oligomers or polymers are terminated with a D-glucose molecule linked to fructose by an α-(1-2) bond.

Inulin is an example of a longer chained compound that is considered a FOS. The shorter (lower molecular weight) compounds tend to have a sweet taste. The size and complexity of the FOS molecule gives it desirable characteristics. Although the simple sugars fructose and glucose are quickly absorbed into the body by the intestines, FOS for the most part is indigestible and therefore acts as a non-digestible fiber in the diet. This is because humans do not have the enzymes to break down the FOS as it travels down the digestive tract. When the FOS reaches the large intestine and the colon, the bacteria that are found there start to break down the FOS. These bacteria have the enzymes needed to break down FOS. Bifido bacteria have been reported to use FOS. It is believed that foods that promote bifido bacteria growth are good for the health.

Both FOS and GOS are indigestible saccharides. β glycosidic linkages of saccharides, such as those found in, but not limited to, FOS and GOS, make these prebiotics mainly indigestible and unabsorbable in the stomach and small intestine. FOS and GOS pass through to the large intestine (colon) mostly intact where they are broken down and metabolized by various probiotics.

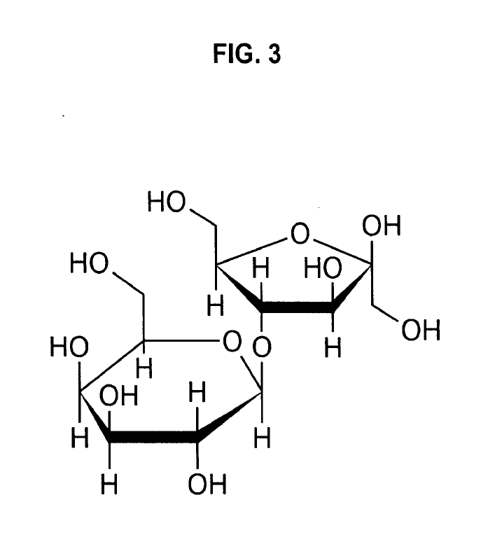

Lactulose (FIG. 3) is a disaccharide formed from one molecule each of fructose and galactose.

Xylo-oligosaccharides (XOS) can be composed of 2-7 xylose molecules connected by β (1-4) glycoside bonds.

In one embodiment the lactose composition with decreased lactose content comprises GOS. In one embodiment the lactose composition with decreased lactose content consists essentially of GOS. In one embodiment the lactose composition with decreased lactose content consists essentially of GOS and further comprises one or more digestible saccharides, such as galactose, or glucose. In one embodiment the lactose composition with decreased lactose content reduces or eliminates a symptom, including but not limited to cramps, flatulance, stomach pain, vomiting bloating, diarrhea, gastric distention and pain, associated with lactose intolerance or with lactose digestive problems.

In one embodiment the lactose composition with decreased lactose content comprises an effective amount of indigestible oligosaccharides. In one embodiment, the indigestible oligosaccharides are galactooligosaccharides. In another embodiment the lactose composition with decreased lactose content comprises GOS, wherein the composition comprises about 0.01%, 0.05%, 0.01% 0.5%, 1%, 2%, 3%, 4%, 5%, 6%, 7%, 8%, 9%, 10%, 11%, 12,%, 13%, 14%, 15%, 16%, 17%, 18%, 19%, 20%, 21%, 22%, 23%, 24%, 25%, 26%, 27%, 28%, 29%, or 30% by weight GOS, and optionally one or more probiotics.

In another embodiment, a serving of the lactose composition with decreased lactose content comprises 0.1-20 g of GOS, such as about 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, or about 20 g of GOS, and optionally one or more probiotics.

In one embodiment the lactose composition with decreased lactose content comprises FOS. In other embodiments, the lactose composition with decreased lactose content comprises about 0.001%, 0.005%, 0.01%, 0.05%, 0.1%, 0.5%, 1%, 2%, 3%, 4%, 5%, 6%, 7%, 8%, 9%, 10%, 11%, 12,%, 13%, 14%, 15%, 16%, 17%, 18%, 19%, 20%, 21%, 22%, 23%, 24%, 25%, 26%, 27%, 28%, 29%, or 30% by weight FOS, and optionally one or more probiotics. In another embodiment, a serving of the lactose composition with decreased lactose content comprises 0.01-20 g of FOS, such as about 0.01, 0.03, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, or 20 g of FOS, and optionally one or more probiotics.

In one embodiment the lactose composition with decreased lactose content comprises inulin, wherein the composition comprises about 0.1%, 0.5%, 1%, 2%, 3%, 4%, 5%, 6%, 7%, 8%, 9%, 10%, 11%, 12,%, 13%, 14%, 15%, 16%, 17%, 18%, 19%, 20%, 21%, 22%, 23%, 24%, 25%, 26%, 27%, 28%, 29%, or 30% by weight inulin, and optionally one or more probiotics. In another embodiment, a serving of the lactose composition with decreased lactose content comprises 1-20 g of inulin, such as about 1, 2, 3, 4, 5, 6, 7, 8, 9, 10, 11, 12, 13, 14, 15, 16, 17, 18, 19, or 20 g of inulin, and optionally one or more probiotics.

In one embodiment the lactose composition with decreased lactose content comprises lactulose, wherein the composition comprises about 0.1%, 0.5%, 1%, 2%, 3%, 4%, 5%, 6%, 7%, 8%, 9%, 10%, 11%, 12,%, 13%, 14%, 15%, 16%, 17%, 18%, 19%, 20%, 21%, 22%, 23%, 24%, 25%, 26%, 27%, 28%, 29%, or 30% by weight lactulose, and optionally one or more probiotics. In another embodiment, a serving of the lactose composition with decreased lactose content comprises 1-20 g of lactulose, such as about 1, 2, 3, 4, 5, 6, 7, 8, 9, 10, 11, 12, 13, 14, 15, 16, 17, 18, 19, or 20 g of lactulose, and optionally one or more probiotics.

In one embodiment the lactose composition with decreased lactose content comprises raffinose, wherein the composition comprises about 0.1%, 0.5%, 1%, 2%, 3%, 4%, 5%, 6%, 7%, 8%, 9%, 10%, 11%, 12,%, 13%, 14%, 15%, 16%, 17%, 18%, 19%, 20%, 21%, 22%, 23%, 24%, 25%, 26%, 27%, 28%, 29%, or 30% by weight raffinose, and optionally one or more probiotics. In another embodiment a serving of the lactose composition with decreased lactose content comprises 1-20 g of raffinose, such as about 1, 2, 3, 4, 5, 6, 7, 8, 9, 10, 11, 12, 13, 14, 15, 16, 17, 18, 19, or 20 g of raffinose, and optionally one or more probiotics.

In one embodiment the lactose composition with decreased lactose content comprises stachyose, wherein the composition comprises about 0.1%, 0.5%, 1%, 2%, 3%, 4%, 5%, 6%, 7%, 8%, 9%, 10%, 11%, 12,%, 13%, 14%, 15%, 16%, 17%, 18%, 19%, 20%, 21%, 22%, 23%, 24%, 25%, 26%, 27%, 28%, 29%, or 30% by weight stachyose, and optionally one or more probiotics. In another embodiment a serving of the lactose composition with decreased lactose content comprises 1-20 g of stachyose, such as about 1, 2, 3, 4, 5, 6, 7, 8, 9, 10, 11, 12, 13, 14, 15, 16, 17, 18, 19, or 20 g of stachyose, and optionally one or more probiotics.

In one embodiment the lactose composition with decreased lactose content comprises XOS, wherein the composition comprises about 0.1%, 0.5%, 1%, 2%, 3%, 4%, 5%, 6%, 7%, 8%, 9%, 10%, 11%, 12,%, 13%, 14%, 15%, 16%, 17%, 18%, 19%, 20%, 21%, 22%, 23%, 24%, 25%, 26%, 27%, 28%, 29%, or 30% by weight XOS, and optionally one or more probiotics. In another embodiment a serving of the lactose composition with decreased lactose content comprises 1-20 g of XOS, such as about 1, 2, 3, 4, 5, 6, 7, 8, 9, 10, 11, 12, 13, 14, 15, 16, 17, 18, 19, or 20 g of XOS, and the lactose composition with decreased lactose content optionally comprises one or more probiotics.

In one embodiment the lactose composition with decreased lactose content comprises TOS, wherein the composition comprises about 0.1%, 0.5%, 1%, 2%, 3%, 4%, 5%, 6%, 7%, 8%, 9%, 10%, 11%, 12,%, 13%, 14%, 15%, 16%, 17%, 18%, 19%, 20%, 21%, 22%, 23%, 24%, 25%, 26%, 27%, 28%, 29%, or 30% by weight TOS, and optionally one or more probiotics. In another embodiment a serving of the lactose composition with decreased lactose content comprises 1-20 g of TOS, such as about 1, 2, 3, 4, 5, 6, 7, 8, 9, 10, 11, 12, 13, 14, 15, 16, 17, 18, 19, or 20 g of TOS, and optionally one or more probiotics.

In one embodiment the lactose composition with decreased lactose content comprises GOS and/or TOS, wherein the GOS and/or molecules are comprised of β-(1-4) glycosidic linkages, and optionally one or more probiotics.

In one embodiment the lactose composition with decreased lactose content comprises GOS and/or TOS, wherein the GOS and/or TOS molecules are comprised of β-(1-6) glycosidic linkages, and optionally one or more probiotics.

In one embodiment the lactose composition with decreased lactose content comprises GOS and/or TOS, wherein the GOS and/or TOS molecules are comprised of β-(1-4) and β-(1-6) glycosidic linkages, and optionally one or more probiotics.

In one embodiment a strain of Bifidobacterium bifidum (accession number NCIMB 41171) produces a galactosidase activity that converts lactose to a galacto-oligosaccharide mixture comprising the disaccharide Gal α (1-6) Gal, at least one trisaccharide selected from Gal β (1-6)-Gal β (1-4)-Glc and Gal β (1-3)-Gal β (1-4)-Glc, the tetrasaccharide Gal β (1-6)-Gal β (1-6)-Gal β (1-4)-Glc and the pentasaccharide Gal β (1-6)-Gal β (1-6)-Gal β (1-6)-Gal β (1-4)-Glc. In one embodiment, the lactose composition with decreased lactose content comprises a GOS composition, wherein the GOS composition comprises a mixture of 20 to 35% w/w of the disaccharide (i.e. the weight of the disaccharide is 20% to 35% the weight of total GOS), 20 to 35% w/w of the trisaccharide, 15 to 25% w/w of the tetrasaccharide and 10 to 20% w/w of the pentasaccharide (see e.g. EP1644482B1).

In another embodiment, the lactose composition with decreased lactose content comprises a GOS composition which comprises a mixture of oligosaccharides comprising 20-28% by weight of β (1-3) linkages (i.e. the weight of the oligosaccharides with β (1-3) linkages is 20-28% the total weight of GOS), 20-25% by weight of 13 (1-4) linkages, and 45-55% by weight of β (1-6) linkages. In one embodiment, a lactose composition with decreased lactose content comprises a GOS composition comprising a mixture of oligosaccharides comprising 26% by weight of β (1-3) linkages (i.e. the weight of the β (1-3) linkages is 26% of the total weight of GOS), 23% by weight of β (1-4) linkages, and 51% by weight of β (1-6) linkages.

In one embodiment the lactose composition with decreased lactose content comprises an effective amount of GOS and optionally another indigestible saccharide to increase Beta-galactosidase activity of a lactobacteria or bifidobacteria strain. In another embodiment the lactose composition with decreased lactose content comprises an effective amount of GOS or another indigestible saccharide to increase the lactase activity of intestinal bacteria (e.g. lactobacteria or bifidobacteria) which breaks down the lactose consumed by a human.

In one embodiment, the lactose composition with decreased lactose content comprises a dairy product and is in the form of milk or other common dairy product such as a yogurt, yogurt drink, shake, smoothie, cheese, and the like.

In one embodiment a lactose composition with decreased lactose content comprises one or more saccharides (herein, interchangeably also referred to as carbohydrate or sugar) which are indigestible by a human digestive system. In another embodiment a lactose composition with decreased lactose content consists essentially of a saccharide which is indigestible by a human digestive system. In one embodiment, the one or more saccharides are oligosaccharides wherein the degree of polymerization is from 2 to 10. In another embodiment, the one or more saccharides are a polysaccharide wherein the degree of polymerization is greater than 10. In another embodiment, the saccharide comprises a mixture of indigestible oligosaccharides or polysaccharides. In another embodiment a lactose composition with decreased lactose content comprises one or more digestable saccharides and one or more indigestible oligosaccharides or polysaccharides. In one embodiment the saccharide is an oligosaccharide, such as a disaccharide, a trisaccharide, a tetrasaccharide, a pentasaccharide, a hexasaccharide, a heptasaccharide, an octasaccharide, a nanasaccharide, or a decasaccharide. Saccharides that are not digestible by humans include, but are not limited to, galacto-oligosaccharides (GOS), transgalacto-oligosaccharide (TOS), lactulose, raffinose, stachyose, lactosucrose, fructo-oligosaccharides (FOS), isomalto-oligosaccharides, xylo-oligosaccharides (XOS), paratinose oligosaccharides, difructose anhydride III, sorbitol, maltitol, lactitol, reduced paratinose, cellulose, β-glucose, β-galactose, β-fructose, verbascose, galactinol, and β-glucan, guar gum, pectin, high sodium alginate, and lambda carrageenan.

In one embodiment the lactose composition with reduced lactose content comprises a saccharide that is inulin, fructo-oligosaccharide (FOS), lactulose, galacto-oligosaccharide (GOS), raffinose, or stachyose. In another embodiment the saccharide that is an oligosaccharide that is indigestible by a human digestive system contains at least one beta-glycosidic (e.g. beta galactosidic or beta glucosidic) bond and when fed to a subject in need thereof would induce lactose digestion. In one embodiment the subject in need thereof is a human. In another embodiment the saccharide is an oligosaccharide that is indigestible by a human digestive system and contains at least one beta-glycosidic (e.g. beta galactosidic or beta glucosidic) bond that can be digested by a bacteria. In one embodiment the bacteria is a probiotic. In one embodiment the bacteria is a lactobacillus or a bifidobacteria. In one embodiment the saccharide is GOS. In another embodiment the saccharide is an oligosaccharide that is indigestible by a human digestive system and contains at least one alpha-glycosidic (e.g. alpha galactosidic or alpha glucosidic) bond that when fed to a subject in need thereof would induce lactose digestion. In one embodiment the subject in need thereof is a human. In another embodiment the saccharide is an oligosaccharide that is indigestible by a human digestive system and contains at least one alpha-glycosidic (e.g. alpha galactosidic or alpha glucosidic) bond that can be digested by a bacterium. In one embodiment the bacteria is a probiotic. In one embodiment the bacterium is a lactobacillus or a bifidobacteria. In one embodiment the saccharide is GOS.

In one embodiment, a lactose composition with decreased lactose content comprising at least one indigestible saccharide optionally contains one or more digestible saccharides or oligosaccharides. In one embodiment, the one or more digestible saccharides are galactose or glucose. In one embodiment, a lactose composition with decreased lactose content does not contain any probiotic bacteria. In another embodiment, a lactose composition with decreased lactose content contains at least one strain of probiotic bacteria.

In one embodiment, a lactose composition with decreased lactose content contains an oligosaccharide that increases β-galatosidase activity in the large intestine. In one embodiment, a lactose composition with decreased lactose content contains an oligosaccharide that increases the amount of probiotic activity in the large intestine.

A lactose composition with decreased lactose content can comprise GOS for improving gut health by promoting the growth of bifidobacteria in the gut. The metabolism of GOS by lactobacilli and bifidobacteria can yield organic acids and other agents that inhibit enteric pathogens. GOS can provide a selective advantage for organisms in the gut that can use them. GOS can also act as anti-adhesives for bacteria in the gut. Thus, a mixture of oligosaccharides are useful for the preparation of a medicament for preventing the adhesion of pathogens or toxins produced by pathogens to the gut wall.

In one embodiment, a lactose composition with decreased lactose content comprises a mixture of one of more of indigestible oligosaccharides, indigestible polysaccharides, free monosaccharides, digestible saccharides, starch, or non-starch polysaccharides.

In one embodiment, a lactose composition with decreased lactose content comprises a GOS composition comprising a mixture of oligosaccharides comprising 1-40% by weight of di-saccharides (i.e. the disaccharides comprise 1-40% of the total weight of GOS), 1-40% by weight tri-saccharides, 1-40% by weight tetra-saccharide, and 1-40% by weight penta-saccharides. In another embodiment, a lactose composition with decreased lactose content comprises a GOS composition comprising a mixture of oligosaccharides consisting essentially of 1-40% by weight of di-saccharides (i.e. the di-saccharides comprise 1-40% of the total weight of GOS), 1-40% by weight tri-saccharides, 1-40% by weight tetra-saccharide, and 1-40% by weight penta-saccharides. In one embodiment, a lactose composition with decreased lactose content comprises a GOS composition comprising a mixture of oligosaccharides comprising 1-40% by weight of saccharides with DP of 1-3 (i.e. the saccharides with DP of 1-3 comprise 1-40% of the total weight of GOS), 1-40% by weight of saccharides with DP of 4-6, 1-40% by weight of saccharides with DP of 7-9, and 1-40% by weight of saccharides with DP of 10-12, 1-40% by weight of saccharides with DP of 13-15.

In another embodiment, a lactose composition with decreased lactose content comprises a GOS composition that comprises a 1:1:1:1:1 ratio of saccharides with a DP of 2:3:4:5:6. In one embodiment, a lactose composition with decreased lactose content comprises a GOS composition comprising a 1:2:3:2:1:1 ratio of saccharides with a DP of 1:2:3:4:5:6. In another embodiment, a lactose composition with decreased lactose content comprises a GOS composition comprising a (12 to 13):(4 to 5):1 ratio of saccharides with a DP of 3:4:5. In one embodiment, a lactose composition with decreased lactose content comprises a GOS composition comprising a 12.3:4.8:1 ratio of saccharides with a DP of 3:4:5. In one embodiment, a lactose composition with decreased lactose content comprises a GOS composition comprising a (8-10):(10-15):(4-6):(1-3) ratio of saccharides with a DP of 2:3:4:5.

In another embodiment, a lactose composition with decreased lactose content comprises a GOS composition comprising a mixture of oligosaccharides comprising 50-55% by weight of di-saccharides (i.e. the di-saccharides comprise 50-55% of the total weight of GOS), 20-30% by weight tri-saccharides, 10-20% by weight tetra-saccharide, and 1-10% by weight penta-saccharides. In one embodiment, a lactose composition with decreased lactose content comprises a GOS composition comprising a mixture of oligosaccharides comprising 52% by weight of di-saccharides (i.e. the di-saccharides comprise 52% of the total weight of GOS), 26% by weight tri-saccharides, 14% by weight tetra-saccharide, and 5% by weight penta-saccharides. In another embodiment, a lactose composition with decreased lactose content comprises a GOS composition comprising a mixture of oligosaccharides comprising 45-55% by weight tri-saccharides (i.e. the tri-saccharides comprise 45-55% of the total weight of GOS), 15-25% by weight tetra-saccharides, 1-10% by weight penta-saccharides. In another embodiment, a lactose composition with decreased lactose content comprises a GOS composition comprising a mixture of oligosaccharides comprising 49.3% by weight tri-saccharides (i.e. the tri-saccharides comprise 49.3% of the total weight of GOS), 19% by weight tetra-saccharides, 4% by weight penta-saccharides.

In another embodiment, a lactose composition with decreased lactose content comprises a GOS composition comprising a mixture of oligosaccharides comprising 2-5% by weight of a mixture of tri- to hexa-saccharides (i.e. tri- to hexa-saccharides comprise 2-5% of the total weight of GOS), 25-35% by weight Galβ (1-6) Glc, 5-15% by weight Galβ (1-3) Glc, 5-15% by weight Galβ (1-2) Glc, 25-30% by weight Galβ (1-6) Gal and 1-5% by weight Galβ (1-3) Gal and optionally further contains one or more digestible saccharides or oligosaccharides. In another embodiment, a lactose composition with decreased lactose content comprises a GOS composition comprising a mixture of oligosaccharides comprising 3.9% by weight of a mixture of tri- to hexa-saccharides (i.e. tri- to hexa-saccharides comprise 3.9% of the total weight of GOS), 32.6% by weight Galβ (1-6) Glc, 7.6% by weight Galβ (1-3) Glc, 9.4% by weight Galβ (1-2) Glc, 27.2% by weight Galβ (1-6) Gal and 2.5% Galβ (1-3) Gal and optionally further contains one or more digestible saccharides or oligosaccharides. In another embodiment, digestible saccharides or oligosaccharides comprise lactose, galactose, or glucose. In one embodiment, a lactose composition with decreased lactose content comprises a GOS composition comprising a mixture of oligosaccharides, lactose, and glucose.

In another embodiment, a lactose composition with decreased lactose content comprises a mixture of FOS and GOS.

Standard analytical methods can be used to determine the amount of the various components of a lactose composition with decreased lactose content, such as but not limited to HPLC, colorimetry (e.g. sodium sulfide colorimetry), spectrophotometry (e.g. atomic absorption spectrophotometry).

In one embodiment, a lactose composition with decreased lactose content comprises a GOS composition comprising a mixture of saccharides that are alpha-GOS and saccharides that are produced by transgalactosylation using β-galactosidase. In another embodiment, GOS comprises alpha-GOS. In another embodiment, alpha-GOS comprises α-(Gal)₂ from 10% to 100% by weight of total GOS. In one embodiment, GOS comprises only saccharides that are produced by transgalactosylation using β-galactosidase.

In one embodiment a lactose composition with decreased lactose content is provided that comprises a suitable amount of prebiotics that are effective for promoting the growth of probiotics such that fermentation in the gut is slowed or such that gastrointestinal health is improved. Numerous prebiotic preparations are known in the art, and any suitable prebiotic preparation can be used in the methods and compositions of the invention. In one embodiment prebiotics can be used in an amount per serving from about 1 mg to about 20 g, about 1 mg to about 15 g, about 1 mg to 10 g, or about 1 mg to about 5 g, or about 2 mg to about 1000 mg, or about 2 mg to about 500 mg, or about 2 mg to about 200 mg, or about 2 mg to about 100 mg, or about 2 mg to about 50 mg, or about 2 mg to about 20 mg, or about 5 mg to about 10 mg, or about 5, 6, 7, 7.5, 8, 9, or 10 mg or about 0.25 g to about 1.7 g. In other embodiments, the prebiotic used can be from about 0.1 g to about 15 g, or about 0.1 g to about 1 g, or about 0.1 g to about 0.5 g or about 0.1 g to about 2 g, or about 0.5 g to about 1 g, or about 0.2 g to about 1 g, or about 1 g to about 5 g per serving or about 1 g to about 15 g per serving. In some embodiments, the smallest effective amount of prebiotic is used. The prebiotic can be about 0.5% to about 20% w/w of the final lactose-reduced dairy composition. In some embodiments, a typical serving size for dairy such as fluid milk is about 240 g. In other embodiments, a serving size is about 245 g, or about 240 g to about 245 g, or about 227 to about 300 g. Yogurt can have a serving size of about 4 oz, or about 6 oz, or about 8 oz, or about 4 oz to 10 oz, or about half cup, or about 1 cup, or about 113 g, or about 170 g, or about 227 g, or about 245 g or about 277 g, or about 100 g to about 350 g.

### Dosage Forms

One aspect provided herein includes methods and compositions comprising formulations for oral delivery to a subject in need thereof. In one embodiment a composition is formulated comprising one or more prebiotics to be delivered a lactose composition with decreased lactose content. In another embodiment a composition is formulated comprising one or more probiotics to be delivered to a lactose composition with decreased lactose content. In another embodiment, a composition is formulated comprising one or more prebiotics and one or more probiotics to be delivered to a lactose composition with decreased lactose content. In one embodiment, one or more prebiotics or probiotics can be formulated to be delivered to a lactose composition with decreased lactose content. In another embodiment, one or more prebiotics or probiotics can be formulated to be co-administered with a lactose composition with decreased lactose content. One or more prebiotics and/or one or more probiotics can be added to a lactose composition with decreased lactose content in a dosage form described below.

A composition can be administered in solid, semi-solid, micro-emulsion, gel, or liquid form. Examples of such dosage forms are known, such as tablet forms disclosed in U.S. Pat. Nos. 3,048,526, 3,108,046, 4,786,505, 4,919,939, 4,950,484; gel forms disclosed in U.S. Pat. Nos. 4,904,479, 6,482,435, 6,572,871, 5,013,726; capsule forms disclosed in U.S. Pat. Nos. 4,800,083, 4,532,126, 4,935,243, 6,258,380; liquid forms disclosed in U.S. Pat. Nos. 4,625,494, 4,478,822, 5,610,184; each of which is incorporated herein by reference in its entirety.

Forms of the compositions that can be used orally include tablets, push-fit capsules made of gelatin, as well as soft, sealed capsules made of gelatin and a plasticizer, such as glycerol or sorbitol. Tablets can be made by compression or molding, optionally with one or more accessory ingredients. Compressed tablets can be prepared by compressing in a suitable machine the active ingredient in a free-flowing form such as a powder or granules, optionally mixed with binders (e.g. povidone, gelatin, hydroxypropylmethyl cellulose), inert diluents, preservative, disintegrant (e.g. sodium starch glycolate, cross-linked povidone, cross-linked sodium carboxymethyl cellulose) or lubricating, surface active or dispersing agents. Molded tablets can be made by molding in a suitable machine a mixture of the powdered compound moistened with an inert liquid diluent. The tablets can optionally be coated or scored and can be formulated so as to provide slow or controlled release of the active ingredient therein. Tablets can optionally be provided with an enteric coating, to provide release in parts of the gut (e.g. colon) other than the stomach. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds (prebiotics or probiotcs) can be dissolved or suspended in suitable liquids, such as fatty oils, liquid paraffin, or liquid polyethylene glycols. In addition, stabilizers can be added. Dragee cores are provided with suitable coatings. For this purpose, concentrated sugar solutions can be used, which can optionally contain gum arabic, talc, polyvinyl pyrrolidone, carbopol gel, polyethylene glycol, or titanium dioxide, lacquer solutions, and suitable organic solvents or solvent mixtures. Dyestuffs or pigments can be added to the tablets or Dragee coatings for identification or to characterize different combinations of active compound doses.

Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water soluble carrier such as polyethylene glycol or an oil medium, for example peanut oil, liquid paraffin, or olive oil.

Oral liquid preparations can be in the form of, for example, aqueous or oily suspensions, solutions, emulsions syrups or elixirs, or can be presented as a dry product for reconstitution with water or other suitable vehicle before use. Such liquid preparations can contain conventional additives, such as suspending agents, for example sorbitol, methyl cellulose, glucose syrup, gelatin, hydroxyethyl cellulose, carboxymethyl cellulose, aluminum stearate gel or hydrogenated edible fats, emulsifying agents, for example lecithin, sorbitan monooleate, acacia; nonaqueous vehicles (which can include edible oils), for example almond oil, oily esters such as glycerine, propylene glycol, or ethyl alcohol; preservatives, for example methyl or propyl p-hydoxybenzoate or sorbic acid, and, if desired, conventional flavoring or coloring agents.

In one embodiment a composition is provided in a dosage form which comprises a an effective amount of one or more prebiotics and/or an effective amount of one or more probiotics, and one or more release controlling excipients as described herein. Suitable modified release dosage vehicles include, but are not limited to, hydrophilic or hydrophobic matrix devices, water-soluble separating layer coatings, enteric coatings, osmotic devices, multi-particulate devices, and combinations thereof. In one embodiment the dosage form is a tablet, caplet, capsule or lollipop. In another embodiment, the dosage form is a liquid, oral suspension, oral solution, or oral syrup. In yet another embodiment, the dosage form is a gel capsule, soft gelatin capsule, or hard gelatin capsule.

In another embodiment a composition comprising an effective amount of one or more prebiotics and/or an effective amount of one or more probiotics is provided in effervescent dosage forms. The compositions can also comprise non-release controlling excipients.

In another embodiment, a composition comprising an effective amount of one or more prebiotics and/or an effective amount of one or more probiotics is provided in a dosage form that has at least one component that can facilitate release of the prebiotic and/or probiotic. In a further embodiment the dosage form can be capable of giving a discontinuous release of a compound in the form of at least two consecutive pulses separated in time from 0.1 up to 24 hours. The compositions can comprise one or more release controlling and non-release controlling excipients, such as those excipients suitable for a disruptable semi-permeable membrane and as swellable substances.

In another embodiment a composition comprising an effective amount of one or more prebiotics and/or an effective amount of one or more probiotics is provided comprising an enteric coated dosage form. The composition can also comprise non-release controlling excipients.

In another embodiment a composition comprising an effective amount of one or more prebiotics and/or an effective amount of one or more probiotics is provided in a dosage form for oral administration to a subject in need thereof, which comprises one or more pharmaceutically acceptable excipients or carriers, enclosed in an intermediate reactive layer comprising a gastric juice-resistant polymeric layered material partially neutralized with alkali and having cation exchange capacity and a gastric juice-resistant outer layer.

In one embodiment a composition comprising an effective amount of one or more prebiotics and/or an effective amount of one or more probiotics is provided in the form of enteric-coated granules, for oral administration. The compositions can further comprise cellulose, disodium hydrogen phosphate, hydroxypropyl cellulose, hypromellose, lactose, mannitol, and sodium lauryl sulfate.

In another embodiment a composition comprising an effective amount of one or more prebiotics and/or an effective amount of one or more probiotics is provided in the form of enteric-coated pellets, for oral administration. The compositions can further comprise glyceryl monostearate 40-50, hydroxypropyl cellulose, hypromellose, magnesium stearate, methacrylic acid copolymer type C, polysorbate 80, sugar spheres, talc, and triethyl citrate.

In one embodiment a composition comprising an effective amount of one or more prebiotics and/or an effective amount of one or more probiotics is provided in the form of enteric-coated granules for oral administration. The compositions can further comprise carnauba wax, crospovidone, diacetylated monoglycerides, ethylcellulose, hydroxypropyl cellulose, hypromellose phthalate, magnesium stearate, mannitol, sodium hydroxide, sodium stearyl fumarate, talc, titanium dioxide, and yellow ferric oxide.

In another embodiment a composition comprising an effective amount of one or more prebiotics and/or an effective amount of one or more probiotics can further comprise calcium stearate, crospovidone, hydroxypropyl methylcellulose, iron oxide, mannitol, methacrylic acid copolymer, polysorbate 80, povidone, propylene glycol, sodium carbonate, sodium lauryl sulfate, titanium dioxide, and triethyl citrate.

The compositions provided herein can be in unit-dosage forms or multiple-dosage forms. Unit-dosage forms, as used herein, refer to physically discrete units suitable for administration to human or non-human animal subject in need thereof and packaged individually. Each unit-dose can contain a predetermined quantity of an active ingredient(s) sufficient to produce the desired therapeutic effect, in association with the required pharmaceutical carriers or excipients. Examples of unit-dosage forms include, but are not limited to, ampules, syringes, and individually packaged tablets and capsules. Unit-dosage forms can be administered in fractions or multiples thereof. A multiple-dosage form is a plurality of identical unit-dosage forms packaged in a single container, which can be administered in segregated unit-dosage form. Examples of multiple-dosage forms include, but are not limited to, vials, bottles of tablets or capsules, or bottles of pints or gallons. In another embodiment the multiple dosage forms comprise different pharmaceutically active agents. For example a multiple dosage form can be provided which comprises a first dosage element comprising a prebiotic and a second dosage element comprising a probiotic, which can be in a modified release form.

In this example a pair of dosage elements can make a single unit dosage. In one embodiment a kit is provided comprising multiple unit dosages, wherein each unit comprises a first dosage element comprising a prebiotic and a second dosage element comprising a probiotic, or both, which can be in a modified release form. In another embodiment the kit further comprises a set of instructions.

In one embodiment compositions can be formulated in various dosage forms for oral administration. The compositions can also be formulated as a modified release dosage form, including immediate-, delayed-, extended-, prolonged-, sustained-, pulsatile-, controlled-, extended, accelerated- and fast-, targeted-, programmed-release, and gastric retention dosage forms. These dosage forms can be prepared according to known methods and techniques (see, Remington: The Science and Practice of Pharmacy, supra; Modified-Release Drug Deliver Technology, Rathbone et al., Eds., Drugs and the Pharmaceutical Science, Marcel Dekker, Inc.: New York, N.Y., 2002; Vol. 126, which is herein incorporated by reference in its entirety).

In one embodiment, the compositions are in one or more dosage forms. For example, a composition can be administered in a solid or liquid form. Examples of solid dosage forms include but are not limited to discrete units in capsules or tablets, as a powder or granule, or present in a tablet conventionally formed by compression molding. Such compressed tablets can be prepared by compressing in a suitable machine the three or more agents and a pharmaceutically acceptable carrier. The molded tablets can be optionally coated or scored, having indicia inscribed thereon and can be so formulated as to cause immediate, substantially immediate, slow, controlled or extended release of a composition comprising an effective amount of one or more prebiotics and/or an effective amount of one or more probiotics. Furthermore, dosage forms of the invention can comprise acceptable carriers or salts known in the art, such as those described in the Handbook of Pharmaceutical Excipients, American Pharmaceutical Association (1986), incorporated by reference herein in its entirety.

In one embodiment, an effective amount of a composition comprising an effective amount of one or more prebiotics and/or an effective amount of one or more probiotics is mixed with a pharmaceutical excipient to form a solid preformulation composition comprising a homogeneous mixture of compounds described herein. When referring to these compositions as “homogeneous”, it is meant that the agents are dispersed evenly throughout the composition so that the composition can be subdivided into unit dosage forms such as tablets, caplets or capsules. This solid preformulation composition can then be subdivided into unit dosage forms of the type described above comprising from, for example, about 1 g to about 20 mg of a prebiotic composition. A composition comprising an effective amount of one or more prebiotics and/or an effective amount of one or more probiotics, can be formulated, in the case of caplets, capsules or tablets, to be swallowed whole, for example with water.

The compositions described herein can be in liquid form. The liquid formulations can comprise, for example, an agent in water-in-solution and/or suspension form; and a vehicle comprising polyethoxylated castor oil, alcohol and/or a polyoxyethylated sorbitan mono-oleate with or without flavoring. Each dosage form comprises an effective amount of an active agent and can optionally comprise pharmaceutically inert agents, such as conventional excipients, vehicles, fillers, binders, disintegrants, pH adjusting substances, buffer, solvents, solubilizing agents, sweeteners, coloring agents and any other inactive agents that can be included in pharmaceutical dosage forms for oral administration. Examples of such vehicles and additives can be found in Remington's Pharmaceutical Sciences, 17th edition (1985).

The dosage forms described herein can be manufactured using processes that are well known to those of skill in the art. For example, for the manufacture of tablets an effective amount of one or more prebiotics and/or an effective amount of one or more probiotics can be dispersed uniformly in one or more excipients, for example, using high shear granulation, low shear granulation, fluid bed granulation, or by blending for direct compression. Excipients include diluents, binders, disintegrants, dispersants, lubricants, glidants, stabilizers, surfactants and colorants. Diluents, also termed “fillers”, can be used to increase the bulk of a tablet so that a practical size is provided for compression. Non-limiting examples of diluents include lactose, cellulose, microcrystalline cellulose, mannitol, dry starch, hydrolyzed starches, powdered sugar, talc, sodium chloride, silicon dioxide, titanium oxide, dicalcium phosphate dihydrate, calcium sulfate, calcium carbonate, alumina and kaolin. Binders can impart cohesive qualities to a tablet formulation and can be used to help a tablet remain intact after compression. Non-limiting examples of suitable binders include starch (including corn starch and pregelatinized starch), gelatin, sugars (e.g. glucose, dextrose, sucrose, lactose and sorbitol), celluloses, polyethylene glycol, waxes, natural and synthetic gums, e.g. acacia, tragacanth, sodium alginate, and synthetic polymers such as polymethacrylates and polyvinylpyrrolidone. Lubricants can also facilitate tablet manufacture; non-limiting examples thereof include magnesium stearate, calcium stearate, stearic acid, glyceryl behenate, and polyethylene glycol. Disintegrants can facilitate tablet disintegration after administration, and non-limiting examples thereof include starches, alginic acid, crosslinked polymers such as, e.g. crosslinked polyvinylpyrrolidone, croscarmellose sodium, potassium or sodium starch glycolate, clays, celluloses, starches, gums and the like. Non-limiting examples of suitable glidants include silicon dioxide, talc and the like. Stabilizers can inhibit or retard drug decomposition reactions, including oxidative reactions. Surfactants can also include and can be anionic, cationic, amphoteric or nonionic. If desired, the tablets can also comprise nontoxic auxiliary substances such as pH buffering agents, preservatives, e.g. antioxidants, wetting or emulsifying agents, solubilizing agents, coating agents, flavoring agents, and the like.

Immediate-release formulations comprising an effective amount of one or more prebiotics and/or an effective amount of one or more probiotics can comprise one or more combinations of excipients that allow for a rapid release of a pharmaceutically active agent (such as from 1 minute to 1 hour after administration). In one embodiment an immediate release excipient can be microcrystalline cellulose, sodium carboxymethyl cellulose, sodium starch glycolate, corn starch, colloidal silica, Sodium Laurel Sulphate, Magnesium Stearate, Prosolve SMCC (HD90), croscarmellose Sodium, Crospovidone NF, Avicel PH200, and combinations of such excipients. “Controlled-release” formulations refers to the release of at least one therapeutic agent from a dosage form at a particular desired point in time after the dosage form has is administered to a subject in need thereof. Generally, controlled-release includes sustained but otherwise complete release. A sudden and total release in the large intestine at a desired and appointed time or a release in the intestines such as through the use of an enteric coating, are both considered controlled-release. Controlled-release can occur at a predetermined time or in a predetermined place within the digestive tract. It is not meant to be a passive, uncontrolled process as in swallowing a normal tablet. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,556; 5,871,776; 5,902,632; and 5,837,284 each of which is incorporated herein by reference in its entirety.

A control release dosage form begins its release and continues that release over an extended period of time. Release can occur beginning almost immediately or can be sustained. Release can be constant, can increase or decrease over time, can be pulsed, can be continuous or intermittent, and the like. Generally, however, the release of at least one pharmaceutically active agent from a controlled-release dosage form will exceed the amount of time of release of the drug taken as a normal, passive release tablet. Thus, for example, while all of at least one pharmaceutically active agent of an uncoated aspirin tablet should be released within, for example, four hours, a controlled-release dosage form could release a smaller amount of aspirin over a period of six hours, 12 hours, or even longer. Controlled-release in accordance with the compositions and methods described herein generally means that the release occurs for a period of six hours or more, such as 12 hours or more.

Extended-release, or sustained-release, refers to the release of an agent, from a composition or dosage form in which the agent is released according to a desired profile over an extended period of time. In one embodiment, controlled-release results in dissolution of an agent within 20-720 minutes after entering the stomach. In another embodiment, controlled-release occurs when there is dissolution of an agent within 20-720 minutes after being swallowed. In another embodiment, controlled-release occurs when there is dissolution of an agent within 20-720 minutes after entering the intestine. In another embodiment, controlled-release results in substantially complete dissolution after at least 1 hour following administration. In another embodiment, controlled-release results in substantially complete dissolution after at least 1 hour following oral administration. For example, controlled-release compositions allow delivery of an agent to a subject in need thereof over an extended period of time according to a predetermined profile. Such release rates can provide therapeutically effective levels of agent for an extended period of time and thereby provide a longer period of pharmacologic or diagnostic response as compared with conventional rapid release dosage forms. Such longer periods of response provide for many inherent benefits that are not achieved with immediate-release dosages. When used in connection with the dissolution profiles discussed herein, the term “controlled-release” refers to wherein all or less than all of the total amount of a dosage form, made according to methods and compositions described herein, delivers an active agent over a period of time greater than 1 hour.

In one aspect, controlled-release refers to delayed release of an agent, from a composition or dosage form in which the agent is released according to a desired profile in which the release occurs after a period of time.

When present in a controlled-release oral dosage form, the compositions described herein can be administered at a substantially lower daily dosage level than immediate-release forms.

In one embodiment, the controlled-release layer is capable of releasing about 30 to about 40% of the one or more active agents (e.g. prebiotic or probiotic) contained therein in the stomach of a subject in need thereof in about 5 to about 10 minutes following oral administration. In another embodiment, the controlled-release layer is capable of releasing about 90% of the one or more active agents (e.g. prebiotic or probiotic) is released in about 40 minutes after oral administration.

In some embodiment, the controlled-release layer comprises one or more excipients, including but not limited to silicified microcrystalline cellulose (e.g. HD90), croscarmellose sodium (AC-Di-Sol), or magnesium stearate. In one embodiment, the total layer weight of the controlled-release layer is from about 100 to about 300 mg, such as about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, or about 300 mg.

In one embodiment, a controlled-release layer comprises from about 75 mg to about 250 mg of silicified microcrystalline cellulose, from about 10 mg to about 40 mg hydroxyl methyl propyl cellulose, from about 0.5 mg to 5 mg magnesium stearate, and from about 0.5 mg to about 5 mg stearic acid.

In one embodiment, the controlled-release layer comprises about 152 mg silicified microcrystalline cellulose, about 20 mg hydroxyl methyl propyl cellulose, about 2.75 mg magnesium stearate, about 2.75 stearic acid.

Pharmaceutical carriers or vehicles suitable for administration of the compounds provided herein include all such carriers known to those skilled in the art to be suitable for the particular mode of administration. In addition, the compositions can include one or more components that do not impair the desired action, or with components that supplement the desired action, or have another action.

In one embodiment, an effective amount of one or more prebiotics and/or an effective amount of one or more probiotics is formulated in an immediate release form. In this embodiment the immediate-release form can be included in an amount that is effective to shorten the time to its maximum concentration in the blood. By way of example, certain immediate-release pharmaceutical preparations are taught in United States Patent Publication US 2005/0147710A1 entitled, “Powder Compaction and Enrobing” which is incorporated herein in its entirety by reference.

The dosage forms described herein can also take the form of pharmaceutical particles manufactured by a variety of methods, including but not limited to high-pressure homogenization, wet or dry ball milling, or small particle precipitation (nano spray). Other methods to make a suitable powder formulation are the preparation of a solution of active ingredients and excipients, followed by precipitation, filtration, and pulverization, or followed by removal of the solvent by freeze-drying, followed by pulverization of the powder to the desired particle size.

In one embodiment the particles have a final size of 3-1000 μM, such as at most 3, 4, 5, 6, 7, 8, 9, 10, 20, 30, 40, 50, 60, 70, 80, 90, 100, 150, 200, 250, 300, 350, 400, 450, 500, 550, 600, 650, 700, 750, 800, 850, 900, 950, 1000 μM. In another embodiment the pharmaceutical particles have a final size of 10-500 μM. In one embodiment the pharmaceutical particles have a final size of 50-600 μM. In another embodiment the pharmaceutical particles have a final size of 100-800 μM.

In a further aspect the dosage form can be an effervescent dosage form. Effervescent means that the dosage form, when mixed with liquid, including water and saliva, evolves a gas. Some effervescent agents (or effervescent couple) evolve gas by means of a chemical reaction which takes place upon exposure of the effervescent disintegration agent to water or to saliva in the mouth. This reaction can be the result of the reaction of a soluble acid source and an alkali monocarbonate or carbonate source. The reaction of these two general compounds produces carbon dioxide gas upon contact with water or saliva. An effervescent couple (or the individual acid and base separately) can be coated with a solvent protective or enteric coating to prevent premature reaction. Such a couple can also be mixed with previously lyophilized particles (such as a prebiotic). The acid sources can be any which are safe for human consumption and can generally include food acids, acid and hydrite antacids such as, for example: citric, tartaric, amalic, fumeric, adipic, and succinics. Carbonate sources include dry solid carbonate and bicarbonate salt such as, preferably, sodium bicarbonate, sodium carbonate, potassium bicarbonate and potassium carbonate, magnesium carbonate and the like. Reactants which evolve oxygen or other gasses and which are safe for human consumption are also included. In one embodiment citric acid and sodium bicarbonate is used.

In another aspect the dosage form can be in a candy form (e.g. matrix), such as a lollipop or lozenge. In one embodiment an effective amount of one or more prebiotics and/or an effective amount of one or more probiotics is dispersed within a candy matrix. In one embodiment the candy matrix comprises one or more sugars (such as dextrose or sucrose). In another embodiment the candy matrix is a sugar-free matrix. The choice of a particular candy matrix is subject to wide variation. Conventional sweeteners such as sucrose can be utilized, or sugar alcohols suitable for use with diabetic patients, such as sorbitol or mannitol might be employed. Other sweeteners, such as the aspartanes, can also be easily incorporated into a composition in accordance with compositions described herein. The candy base can be very soft and fast dissolving, or can be hard and slower dissolving. Various forms will have advantages in different situations.

A candy mass composition comprising an effective amount of the prebiotic can be orally administered to a subject in need thereof so that an effective amount of the prebiotic will be released into the subject's mouth as the candy mass dissolves and is swallowed. A subject in need thereof includes a human adult or child.

In one embodiment a candy mass is prepared that comprises one or more layers which can comprise different amounts or rates of dissolution of a composition comprising an effective amount of one or more prebiotics and/or an effective amount of one or more probiotics. In one embodiment a multilayer candy mass (such as a lollipop) comprises an effective amount of one or more prebiotics and/or an effective amount of one or more probiotics differing from that of one or more inner layers. Such a drug delivery system has a variety of applications.

The choices of matrix and the concentration of the drug in the matrix can be important factors with respect to the rate of drug uptake. A matrix that dissolves quickly can deliver drug into the subject in need thereof's mouth for absorption more quickly than a matrix that is slow to dissolve. Similarly, a candy matrix that contains an effective amount of one or more prebiotics and/or an effective amount of one or more probiotics in a high concentration can release more of an effective amount of one or more prebiotics and/or an effective amount of one or more probiotics in a given period of time than a candy having a low concentration. In one embodiment a candy matrix such as one disclosed in U.S. Pat. No. 4,671,953 or US Application 2004/0213828 (which are herein incorporated by reference in their entirety) is used to deliver an effective amount of one or more prebiotics and/or an effective amount of one or more probiotics.

The dosage forms described herein can also take the form of pharmaceutical particles manufactured by a variety of methods, including but not limited to high-pressure homogenization, wet or dry ball milling, or small particle precipitation (e.g. nGimat's NanoSpray). Other methods useful to make a suitable powder formulation are the preparation of a solution of active ingredients and excipients, followed by precipitation, filtration, and pulverization, or followed by removal of the solvent by freeze-drying, followed by pulverization of the powder to the desired particle size. In one embodiment the pharmaceutical particles have a final size of 3-1000 μM, such as at most 3, 4, 5, 6, 7, 8, 9, 10, 20, 30, 40, 50, 60, 70, 80, 90, 100, 150, 200, 250, 300, 350, 400, 450, 500, 550, 600, 650, 700, 750, 800, 850, 900, 950, 1000 μM. In another embodiment the pharmaceutical particles have a final size of 10-500 μM. In another embodiment the pharmaceutical particles have a final size of 50-600 μM. In another embodiment the pharmaceutical particles have a final size of 100-800 μM.

Compositions described herein include any suitable form, including liquid, powder, or freeze dried powder. Powdered compositions can be as pure powder, or can be in the form of capsules, tablets, or the like. Powder can be packaged in bulk (e.g. in a container containing sufficient prebiotic or other substances for a subject in need thereof to follow for an entire course of treatment with increasing doses of prebiotic, or a portion of a course of treatment), or as individual packets (e.g. packets containing a single dose of prebiotic plus other components, or packets containing the dose of prebiotic and other components needed for a particular day of a prebiotic treatment regimen). If packaged in bulk, the powder can be in any suitable container, such as a packet, sachet, canister, ampoule, ramekin, or bottle. The container can also include one or more scoops or similar serving devices of a size or sizes appropriate to measure and serve one or more doses of prebiotic and, optionally, other ingredients included in the powder. Liquid compositions contain prebiotic and, optionally, other ingredients, in a suitable liquid, e.g. water or buffer. Liquid compositions can be provided in bulk (e.g. in a container containing sufficient prebiotic or other substances for one subject in need thereof to follow an entire course of treatment with increasing doses of prebiotic, or a portion of a course of treatment), or as individual containers, such as cans, bottles, soft packs, and the like (e.g. containers containing a single dose of prebiotic plus other components in suitable liquid, or containers containing the dose of prebiotic and other components needed for a particular day of a prebiotic treatment regimen). The container can also include one or more measuring cups or similar serving devices of a size or sizes appropriate to measure and serve one or more doses of prebiotic and, optionally, other ingredients included in the liquid.

### Formulations

In one embodiment a lactose composition with decreased lactose content comprises inulin, fructo-oligosaccharide (FOS), lactulose, galacto-oligosaccharide (GOS), raffinose, stachyose, or a combination thereof. In one embodiment a lactose composition with decreased lactose content comprises or consists essentially of GOS. In another embodiment a lactose composition with decreased lactose content contains a GOS. In another embodiment a lactose composition with decreased lactose content contains GOS and at least one probiotic bacteria strain. Additional ingredients include ingredients to improve handling, preservatives, flavorings and the like.

In one embodiment, a lactose composition with decreased lactose content comprises GOS and at least one probiotic bacteria strain. Any remaining ingredients can be any suitable ingredients intended for the consumption of the subject in need thereof, e.g. human, including, but not limited to, other prebiotics (e.g. FOS), a buffer, digestible saccharides (e.g. glucose or galactose), ingredients intended to inhibit clumping and increase pourability, such as silicone dioxide and microcrystalline cellulose, or similar ingredients as are well-known in the art. Remaining ingredients can also include ingredients to improve handling, preservatives, flavorings and the like.

In one embodiment, a lactose composition with decreased lactose content comprises lactose and GOS. In one embodiment, lactose is present at about 5% by weight. Any remaining ingredients can be any suitable ingredients intended for the consumption of the subject in need thereof, e.g. human, including, but not limited to, digestible saccharides (e.g. glucose or galactose, bacteria a buffer, ingredients intended to inhibit clumping and increase pourability, such as silicone dioxide and microcrystalline cellulose, or similar ingredients as are well-known in the art). Remaining ingredients can also include ingredients to improve handling, preservatives, flavorings and the like.

In one embodiment, a lactose composition with decreased lactose content comprises lactose, bacteria (e.g. L. acidophilus), and GOS. In one embodiment, lactose can be present at about 1-20% by weight and bacteria at about 0.25-2.10% by weight. Any remaining ingredients can be any suitable ingredients intended for the consumption of the subject in need thereof, e.g. human, including, but not limited to a buffer, digestible saccharides (e.g. glucose or galactose) intended to inhibit clumping and increase pourability, such as silicone dioxide and microcrystalline cellulose, or similar ingredients as are well-known in the art. Remaining ingredients can also include ingredients to improve handling, preservatives, flavorings and the like.

Additional ingredients include ingredients to improve handling, preservatives, flavorings and the like. For example, in one embodiment, a prebiotic composition in powdered form can include flavorings such that when mixed in a liquid (e.g. water), liquid can have various flavors such as grape, strawberry, lime, lemon, chocolate, and the like. In one embodiment, the compositions include microcrystalline cellulose and silicone dioxide.

### Other Components

One or more buffers, optionally with a calcium counterion, can also be administered in methods and compositions described herein. Any buffer suitable for consumption by the subject in need thereof being treated, e.g. human, are useful for the compositions herein. The buffer neutralizes stomach acidity which can, e.g. allow live bacteria to reach the gut. Buffers include citrates, phosphates, and the like. One embodiment utilizes a buffer with a calcium counterion, such as Calcium Phosphate Tribasic. The calcium can serve to restore the calcium that many lactose intolerant subjects are missing in their diet. A recent study demonstrated the ability of calcium phosphate to protect lactobacillus acidophilus from bile. It is an excellent buffering agent and will help neutralize stomach acidity.

Numerous buffers suitable for human consumption are known in the art, and any suitable buffer can be used in the methods and compositions described herein. Calcium triphosphate is an exemplary buffer and has the advantage that its counterion supplies a nutrient that is often lacking in lactose-intolerant subjects in need thereof, i.e., calcium. The buffer can be used in a dose from about 2 to about 2000 mg, or about 4 to about 400 mg, or about 4 to about 200 mg, or about 4 to about 100 mg, or about 8 to about 50 mg, or about 10 to about 40 mg, or about 20 to about 30 mg, or about 20, 21, 22, 23, 24, 25, 26, 27, 28, 29, or 30 mg. In one embodiment, buffer is used in a dose of about 25 mg. In one embodiment, calcium phosphate is used in a dose of about 25 mg.

### General Considerations

In some embodiments, the lactose composition with decreased lactose content can contain one or more prebiotics and no probiotics. In some embodiments, the lactose composition with decreased lactose content can contain one or more probiotics and no prebiotics. In other embodiments, the lactose composition with decreased lactose content can contain both one or more prebiotics and one or more probiotics.

The lactose composition with decreased lactose content described can be used prior to, in conjunction with, or after following other regimens developed for reducing lactose intolerance (e.g., U.S. Pat. No. 7,029,702, US Publication No. 2008/0126195). Regular dosing of the probiotics and/or prebiotics can help supplement or prolong the results from these regimens, for example through colonic adaptation.

The lactose compositions with decreased lactose content can also be useful for improving overall GI health. Probiotics, such as Lactobacilli and Bifidobacteria, help support a healthful and balanced population of intestinal bacteria. When the GI tract does not function properly, important nutrients are not easily absorbed and can affect the body's systems. An individual's energy levels, moods, weight, skin, joints, mental acuity, and respiratory function can be affected, allowing the individual to become prone to more serious conditions. A weakened or compromised GI lining has been shown to play a role in inflammatory bowel disease (IBD), ulcers, and various forms of hepatitis (Galperin C, Gershwin M E. Immunopathogenesis of gastrointestinal and hepatobiliary diseases. JAMA 1997 Dec. 10; 278(22):1946-55).

The lactose composition with decreased lactose content comprising one or more probiotics and/or prebiotics can be produced in multiple forms such as single serve packages or multiple serving packages.

At any stage of the production of the lactose composition with decreased lactose content, one or more probiotics can be added as long as the bacteria are not inactivated during processing. At any stage of the production of the lactose compositions with decreased lactose content, one or more prebiotics can be added as long as the prebiotic remains a viable energy source after processing.

One or more additional ingredients can be used in the compositions such as ingredients to improve handling, preservatives, flavorings, buffers, and the like.

### Business Methods

The invention also provides business methods for marketing compositions and methods for the treatment of the symptoms of lactose intolerance or for overall improvement of in gastrointestinal health. In one embodiment, the invention provides a method of doing business that includes marketing a composition for the treatment of symptoms of lactose intolerance or for overall improvement of gastrointestinal health, wherein the treatment or improvement are brought about by consumption of lactose-reduced dairy products supplemented with one or more probiotics and/or one or more prebiotics according to any of the methods described herein. The methods can further include producing such compositions. The marketing can be directly to the consumer, or to suitable health professionals, or combinations thereof. The methods of marketing used in these embodiments of the invention include, but are not limited to, print, television, or radio commercials, infomercials, internet advertising, testimonials, word of mouth, telemarketing, and the like.

The examples described herein are not intended to be limiting, but merely illustrative of the forms in which the invention can be used as part of a method for reducing symptoms associated with lactose intolerance in mammals or as part of a method for overall improvement of gastrointestinal health.

### EXAMPLES

The following are examples to illustrate some embodiments described herein.

### Example 1

Milk with decreased lactose is supplemented with probiotics by adding about 1×10⁹ cfu's of Lactobacillus acidophilus to one cup of milk with decreased lactose such that lactose content is about 0.01% to about 5% (w/w) lactose.

### Example 2

Milk with decreased lactose is supplemented with probiotics by adding about 1.25×10⁸ cfu's of Bifidobacterium longum to one cup of milk with decreased lactose such that lactose content is about 0.01% to about 5% (w/w) lactose.

### Example 3

Milk with decreased lactose is supplemented with probiotics by adding about 8.25×10⁶ cfu's of Bifidobacterium bifidum to one cup of milk with decreased lactose such that lactose content is about 0.01% to about 5% (w/w) lactose.

### Example 4

Milk with decreased lactose is supplemented with prebiotics by adding about 1 g of fructo-oligosaccharide to one cup of milk with decreased lactose such that lactose content is about 0.01% to about 5% (w/w) lactose.

### Example 5

Milk with decreased lactose is supplemented with prebiotics by adding about 0.33 g of fructo-oligosaccharide to one cup of milk with decreased lactose such that lactose content is about 0.01% to about 5% (w/w) lactose.

### Example 6

Milk with decreased lactose is supplemented with prebiotics by adding about 0.33 g of fructo-oligosaccharide and about 3.25×10⁸ cfu's of Lactobacillus acidophilus to one cup of milk with decreased lactose such that lactose content is about 0.01% to about 5% (w/w) lactose.

### Example 7

Yogurt with decreased lactose content such that lactose content is about 0.01% to about 7.5% (w/w) lactose, in various flavors such as vanilla, strawberry, mixed berry, prune, peach, and blueberry, is supplemented with probiotic by adding about 10⁵ to about 10⁶ cfu of B. animalis per gram yogurt. This represents about 113×10⁵ cfu's to about 113×10⁶ cfu's per 113 g serving of yogurt.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein can be employed in practicing the invention. It is intended that the following claims define the scope of the invention and that methods and structures within the scope of these claims and their equivalents be covered thereby.

1-35. (canceled)

36. A method of treating pain comprising administering to a subject in need thereof a pharmaceutical formulation comprising a lactose composition with decreased lactose content comprising an effective amount of galacto-oligosaccharides, another indigestible saccharide, and one or more pharmaceutically acceptable excipients or carriers.

37. The method of claim 36, wherein the pain is associated with lactose intolerance.

38. The method of claim 36, wherein the another indigestible saccharide is lactulose, raffinose, stachyose, lactosucrose, fructo-oligosaccharides, isomalto-oligosaccharides, xylo-oligosaccharides, paratinose oligosaccharides, difructose anhydride III, sorbitol, maltitol, lactitol, reduced paratinose, cellulose, β-glucose, β-galactose, β-fructose, verbascose, galactinol, β-glucan, guar gum, pectin, high sodium alginate, or lambda carrageenan.

39. The method of claim 36, wherein the pharmaceutical formulation further comprises at least one probiotic bacteria strain.

40. The method of claim 39, wherein the probiotic bacteria strain is from the genera Lactobacilus or Bifidobacteria.

41. The method claim 39, wherein the pharmaceutical formulation comprises from 1×10⁶ to 1×10⁹ colony forming units (cfu) of the probiotic bacteria strain.

42. The method of claim 36, wherein the pharmaceutical formulation comprises 0.1-20 g per serving of the galactooligosaccharides.

43. The method of claim 36, wherein the subject in need thereof experiences a decrease in at least one symptom of lactose intolerance after administration.

44. The method of claim 43, wherein the symptom of lactose intolerance is gas, bloating, flatulence, diarrhea, abdominal pain, cramping, stomach pain, or vomiting.

45. The method claim 36, wherein the lactose composition with decreased lactose content comprises from 0.001% to 5.3% lactose.

46. The method claim 36, wherein the pharmaceutical formulation is in the form of a tablet, capsule, powder, effervescent dosage form, or candy form.

47. A pharmaceutical formulation suitable for oral administration comprising a lactose composition with decreased lactose content comprising an effective amount of galactooligosaccharides for treating pain, another indigestible saccharide, and one or more pharmaceutically acceptable excipients or carriers.

48. The pharmaceutical formulation of claim 47, wherein the pain is associated with lactose intolerance.

49. The pharmaceutical formulation of claim 47, wherein the another indigestible saccharides is lactulose, raffinose, stachyose, lactosucrose, fructo-oligosaccharides, isomalto-oligosaccharides, xylo-oligosaccharides, paratinose oligosaccharides, difructose anhydride III, sorbitol, maltitol, lactitol, reduced paratinose, cellulose, β-glucose, β-galactose, β-fructose, verbascose, galactinol, β-glucan, guar gum, pectin, high sodium alginate, or lambda carrageenan.

50. The pharmaceutical formulation of claim 47, wherein the pharmaceutical formulation further comprises at least one probiotic bacteria strain.

51. The pharmaceutical formulation of claim 50, wherein the probiotic bacteria strain is from the genera Lactobacilus or Bifidobacteria.

52. The pharmaceutical formulation claim 50, wherein the pharmaceutical formulation comprises from 1×10⁶ to 1×10⁹ colony forming units (cfu) of the probiotic bacteria strain.

53. The pharmaceutical formulation of claim 47, wherein the pharmaceutical formulation comprises from 1 mg to 20 g per serving of the galactooligosaccharides and another indigestible saccharides.

54. The pharmaceutical formulation claim 47, wherein the lactose composition with decreased lactose content comprises from 0.001% to 5.3% lactose.

55. The orally administered pharmaceutical formulation claim 47, wherein the pharmaceutical formulation is in the form of a tablet, capsule, powder, effervescent dosage form, or candy form.
